(19) United States
(12) Patent Application Publication
(10) Pub. No.: US 2020/0297654 A1
(43) Pub. Date: Sep. 24, 2020

(54) COMPOSITION AND DEVICE FOR DELIVERY OF ACTIVE AGENTS TO SKIN SURFACES

(71) Applicant: Lubrizol Advanced Materials, Inc., Cleveland, OH (US)

(72) Inventors: Nancy S. Marchant, Medina, OH (US); Thomas Beckert, Warthausen (DE); Jürgen Heinzelmann, Untermarchtal (DE); Anja Thut, Feucht (DE); Jochen Stehle, Biberach (DE); Qiong Tang, Cleveland, OH (US); Hossein Birjandi Nejad, McLean, VA (US); Mohamed Hussein Ramadan, Cleveland, OH (US)

(73) Assignee: Lubrizol Advanced Materials, Inc., Cleveland, OH (US)

(21) Appl. No.: 16/756,366

(22) PCT Filed: Oct. 16, 2018
(86) PCT No.: PCT/US2018/056073

§ 371 Date: Apr. 15, 2020

Related U.S. Application Data
(60) Provisional application No. 62/573,225, filed on Oct. 17, 2017.

(51) Int. Cl. A61K 9/70; A61K 31/4468; A61K 31/167; A61K 31/045

(52) U.S. Cl. CPC A61K 9/7069; A61K 9/7084; A61K 31/045; A61K 31/167; A61K 31/4468

(57) ABSTRACT

A transdermal delivery device (10, 10′) includes at least one of a backing layer (14) and a removable release liner (16). An active layer (12, 12′) is supported by the backing layer and/or release liner. The active layer includes a polymer matrix, a therapeutically/cosmetically effective amount of an active agent dispersed in the polymer matrix, and a pressure sensitive adhesive, incorporated in the polymer matrix and/or adhered thereto. The polymer matrix includes a thermoplastic polyurethane polymer and optionally, a poly(meth)acrylate polymer. The thermoplastic polyurethane polymer includes the reaction product of: a first polyether polyol A having a molecular weight of at least 3000 daltons and/or a second polyether polyol B having a molecular weight of no more than 2500 daltons; a third polyol C having a molecular weight of up to 800 daltons and/or a chain extender; a polyisocyanate; and optionally a catalyst.

This application claims the priority of International Application PCT/US2018/056073, filed Oct. 16, 2018, and U.S. Provisional Application Ser. No. 62/573,225, filed Oct. 17, 2017, from which the PCT application claims priority, the disclosures of which are incorporated herein in their entireties by reference.

### BACKGROUND

Aspects of the exemplary embodiment relate to compositions and drug delivery devices for transdermal and/or topical application of active agents, methods of making them, and methods of effecting transdermal and/or topical delivery of active agents using the same.

Many factors influence the design and performance of transdermal and/or topical drug delivery compositions and devices. These include the individual drugs themselves, the physical and chemical characteristics of the compositions' components and their performance and behavior relative to other components, external and environmental conditions during manufacturing and storage, properties of the application site, the desired rate of drug delivery and therapeutic onset, the desired drug delivery profile, and the intended duration of delivery, among others.

Compositions for the transdermal and/or topical delivery of drugs are known. For example, U.S. Pub. No. 20150352058, describes a transdermal drug delivery system including a backing layer, a drug-containing matrix layer, and a release layer. The matrix layer includes an acrylate-rubber hybrid as an adhesive. U.S. Pub. No. 20050048104 describes drug reservoirs including a polyurethane polymer which can be processed at temperatures below those which cause degradation of temperature sensitive drugs and/or excipients. Compositions and patches for percutaneous fentanyl administration are described in U.S. Pat. Nos. 7,785,622, 7,718,188 and 9,295,655, US Pub. No. 20060222691, and WO2009051217 A1. U.S. Pat. No. 9,265,752, to Wang, et al., describes a patch containing a Rotigotine-containing composition. WO2014151492 A1, to Noven Pharma, describes a transdermal drug delivery system including a polymer matrix including the free base form of a tertiary amine drug and at least one carboxyl group-containing compound to form a salt. Matrix films for delivery of repaglinide are described in Vijaya, et al., “Development and in vitro evaluation of Eudragit E100 and PVP based matrix films for the transdermal delivery of Repaglinide,” The Pharma Innovation Journal, 3(2): 16-23 (2015).

There remains a need for compositions that exhibit suitable physical and pharmacokinetic properties. More specifically there is a need for compositions that allow for acceptable drug release while also providing the good adhesion to the skin or other surface where the composition is being used for the duration of its use while at the same time being readily removable without causing undue damage and/or discomfort to the skin or other surface where the composition is being used.

### SUMMARY

Aspects of the exemplary embodiment relate to devices, compositions and methods for delivery of active agents to skin surfaces.

In a first aspect, a transdermal delivery device includes or consists essentially of at least one of a backing layer and a removable release liner; and an active layer supported by the at least one of the backing layer and the removable release liner. The active layer includes or consists essentially of: a polymer matrix, a therapeutically or cosmetically effective amount of an active agent dispersed in the polymer matrix, and a pressure sensitive adhesive, the pressure sensitive adhesive being incorporated in at the polymer matrix and/or adhered to the polymer matrix. The polymer matrix includes or consists essentially of (a) a thermoplastic polyurethane polymer which is the reaction product of (1) at least one of: a first polyether polyol A having a molecular weight of at least 3000 daltons and a second polyether polyol B having a molecular weight of no more than 2500 daltons; (2) at least one of: a third polyol C having a molecular weight of up to 800 daltons and a chain extender; (3) a polyisocyanate; (3) optionally, a catalyst; (4) optionally at least a portion of the pressure sensitive adhesive; the polymer matrix optionally including a poly(meth)acrylate polymer.

In various embodiments, the device further includes one or more of the following:
the pressure sensitive adhesive is incorporated in the polymer matrix.
the pressure sensitive adhesive is at least 20 wt. % of the polymer matrix, or at least 30 wt. %, or up to 80 wt. % or up to 70 wt. % of the polymer matrix.
the thermoplastic polyurethane polymer is at least 20 wt. % of the polymer matrix, or up to 70 wt. % of the polymer matrix.
the pressure sensitive adhesive is in a layer which is in contact with a layer containing the polymer matrix.
the pressure sensitive adhesive is selected from the group consisting of acrylic-based pressure sensitive adhesives and silicone-based pressure sensitive adhesives.
the pressure sensitive adhesive comprises at least one vinyl acetate acrylic pressure sensitive adhesive.
the poly(meth)acrylate polymer is present in the active layer.
the poly(meth)acrylate polymer is selected from the group consisting of: poly(butyl methacrylate-co-(2-dimethylaminoethyl) methacrylate-co-methyl methacrylate); poly(methacrylic acid-co-methyl methacrylate); poly(ethyl acrylate-co-methyl methacrylate-co-trimethylaminoethyl methacrylate chloride); poly(methacrylic acid-co-methyl methacrylate); poly(methacrylic acid-co-ethyl acrylate), and mixtures thereof.
a ratio by weight of poly(meth)acrylate polymer:thermoplastic polyurethane polymer in the polymer matrix is at least 1:100, or at least 1:20, or at least 1:5.
the poly(meth)acrylate is a cationic copolymer derived from dimethylaminoethyl methacrylate, butyl methacrylate, and methyl methacrylate.
the poly(meth)acrylate is at least 2 wt. % of the polymer matrix, or at least 5 wt. %, or at least 10 wt. %, or up to 50 wt. %, or up to 45 wt. %, or up to 40 wt. %.
the active layer is no more than 10 wt. % water, or no more than 3 wt. water, or no more than 1 wt. % water.
the thermoplastic polyurethane polymer has a hard segment content of at least 4 wt. % and a soft segment content of at least 90 wt. %.
the chain extender is selected from 1,4-butanediol, 1,10-decanediol, and combinations thereof.
at least one of: the first polyether polyol A has a molecular weight of at least 4000 daltons; the second polyether polyol B, when present, has a molecular weight of no more than 2000 daltons; a ratio of the molecular weight of the first polyether polyol to the second polyether polyol, when the second polyether polyol is present, is at least 1.4:1; a ratio by weight of the second polyether polyol to the first polyether polyol, when the second polyether is present, is at least 1:1; and the chain extender has a molecular weight of up to 300 daltons.
the polyisocyanate is selected from cycloaliphatic diisocyanates and aromatic diisocyanates.
the active layer has a thickness of up to 1 mm.
the active agent is selected from the group consisting of agomelatine, amphetamine, scopolamine, levonorgestrel, ethinyl estradiol, levonorgestrel acetate, testosterone, methylphenidate, enalaprilat ethyl ester, capsaicin, clonidine, ketoprofen, nicotine, lidocaine, lidocaine HCL, fentanyl, rivastigmine, pharmaceutically acceptable salts, prodrugs, and/or derivatives thereof, and combinations thereof.
the thermoplastic polyurethane polymer includes the reaction product of: (i) the first polyether polyol A, which comprises a poly(ethylene glycol) with an average molecular weight of 7000-9000 daltons; (ii) the second polyether polyol B, which comprises a polyether copolymer diol with polypropylene glycol and polyethylene oxide segments and an average molecular weight of about 2000 daltons; (iii) the at least one of the third polyol and the chain extender; (iv) optionally, one or more additional polymer forming components; and (v) the polyisocyanate.
the chain extender comprises at least one of 1,4-butanediol and 1,10-decanediol.
the polyisocyanate comprises dicyclohexylmethane 4,4′ diisocyanate.
the thermoplastic polyurethane polymer includes the reaction product of: optionally, the first polyether polyol A; the second polyether polyol B, which includes a polyether copolymer diol with polypropylene glycol and polyethylene oxide segments and an average molecular weight of 1800-2200 daltons; (iii) the chain extender; (iv) optionally, one or more additional polymer forming components; and (v) the polyisocyanate.
the thermoplastic polyurethane polymer includes the reaction product of, (totaling 100 wt. %): 20-30 wt. % Polyol A; 40-70 wt. % Polyol B; 5-20 wt. polyisocyanate; 0.2-5 wt. % Polyol C; 0-5 wt. % chain extender; optionally, one or more additional polymer forming components; and optionally up to 2 wt. % of a catalyst.
the thermoplastic polyurethane polymer comprises the reaction product of (totaling 100 wt. %): 70-90 wt. % Polyol A; no more than 5 wt. % Polyol B; 5-20 wt. polyisocyanate; 0-15 wt. % Polyol C; 0-10 wt. % chain extender; optionally, one or more additional polymer forming components; and optionally, up to 2 wt. % of a catalyst.
the thermoplastic polyurethane polymer comprises the reaction product of (totaling 100 wt. %): no more than 5 wt. % Polyol A; 30-90 wt. % Polyol B; 5-50 wt. polyisocyanate; 0-5 wt. % Polyol C; 0-20 wt. % chain extender; optionally, one or more additional polymer forming components, and optionally up to 2 wt. % of a catalyst.
the active layer further includes a penetration enhancer.
the active layer is adhered to the backing layer.
the active layer is adhered to one of the adhesive layer and the release liner.

A method for the transdermal delivery of an active pharmaceutical ingredient includes topically applying the device as described in any of the above aspects, after removal of the release liner, where present, to the skin of a subject in need thereof.

In another aspect of the exemplary embodiment, a method for the manufacture of a transdermal delivery device includes or consists essentially of: forming or otherwise providing a composition and forming a multilayer structure in which the composition forms a layer intermediate a backing layer and a release liner, whereby the pressure sensitive adhesive is present in at least one of the active layer and a separate layer which spaces the active layer from the release liner. The composition includes or consists essentially of a therapeutically or cosmetically effective amount of an active pharmaceutical or cosmetic ingredient; optionally, a poly(meth)acrylate polymer; a pressure sensitive adhesive; and at least 20 wt. % of a thermoplastic polyurethane polymer. The thermoplastic polyurethane polymer includes or consists essentially of the reaction product of: at least one of a first polyether polyol A having a molecular weight of at least 3000 daltons and a second polyether polyol B having a molecular weight of no more than 2500 daltons; at least one of a third polyol C having a molecular weight of up to 800 daltons and a chain extender; a polyisocyanate; and optionally, a catalyst;

In another aspect of the exemplary embodiment, which may further include any of the aspects described above, a composition for the delivery of an active pharmaceutical ingredient in the form of a flexible finite device for topical application is provided. The composition includes a polymer matrix including or consisting essentially of: at least 20 wt. % of a thermoplastic polyurethane polymer, at least 30 wt. % of a pressure sensitive adhesive; and at least 2 wt. % of a poly(methacrylate). The composition further includes a therapeutically or cosmetically effective amount of an active pharmaceutical or cosmetic ingredient dispersed in the polymer matrix. The thermoplastic polyurethane polymer includes the reaction product of: a first polyether polyol having a molecular weight of at least 3000 daltons; a second polyether polyol having a molecular weight of no more than 2500 daltons; at least one of a third polyol having a molecular weight of up to 800 daltons and a chain extender; a polyisocyanate; and optionally, a catalyst.

In another aspect of the exemplary embodiment, which may further include any of the aspects described above, a transdermal delivery device includes or consists essentially of (i) a backing layer; (ii) an integrated matrix including: (a) a therapeutically or cosmetically effective amount of an active pharmaceutical/cosmetic ingredient; and (b) 20 to 70 wt. % of a thermoplastic polyurethane polymer which includes or consists essentially of the reaction product of: (i) a first polyether polyol having a molecular weight of at least 3000 daltons; (ii) optionally, a second polyether polyol having a molecular weight of no more than 2500 daltons; (iii) at least one of: a third polyol having a molecular weight of up to 800 daltons and a chain extender; (iv) a polyisocyanate; and (v) optionally, a catalyst; and (c) 30 to 80 wt. % of a pressure sensitive adhesive; the combined adhesive and drug-containing carrier layer having a first surface and a second surface, the first surface of the integrated matrix being directly affixed to a surface of the backing layer; and (iii) a removable release liner, the removable release liner being directly affixed to the second surface of the integrated matrix.

### BRIEF DESCRIPTION OF THE DRAWINGS

FIG. 1 is a side sectional view of a topical or transdermal delivery device in accordance with a first aspect of the exemplary embodiment;

FIG. 2 is a side sectional view of the topical or transdermal delivery device of FIG. 1 on a skin surface;

FIG. 3 is a side sectional view of a topical or transdermal delivery device in accordance with a second aspect of the exemplary embodiment;

FIG. 4 illustrates a method for forming the device of FIG. 1, in accordance with another aspect of the exemplary embodiment;

FIG. 5 is a plot showing the absolute permeation of fentanyl from a TPU/Acrylic Adhesives blend and from a reference fentanyl product (Control 1);

FIG. 6 is a plot showing absolute permeation of fentanyl from a TPU/Acrylic Adhesive Blend, with and without Eudragit® E100, and from the reference product (Control 1);

FIG. 7 is a plot showing relative permeation across mouse skin of example formulations containing fentanyl; and

FIG. 8 is a plot showing absolute permeation across mouse skin of example formulations containing fentanyl.

### DETAILED DESCRIPTION

Various preferred features and embodiments will be described below by way of non-limiting illustration.

Described herein are improved drug compositions that provide increased stability, higher drug loading potential, and suitable drug dissolution properties, devices which include them, and methods of making the same.

Technical and scientific terms used herein have the meanings commonly understood by one of ordinary skill in the art, unless defined otherwise. Reference is made herein to various methodologies known to those of ordinary skill in the art, as well as publications and other materials setting forth such known methodologies, all of which are incorporated herein by reference in their entireties. Any suitable materials and/or methods known to those of ordinary skill in the art can be utilized in carrying out the present invention. However, specific materials and methods are described. Materials, reagents and the like to which reference is made in the following description and examples are obtainable from commercial sources, unless otherwise noted.

As used herein, the singular forms “a,” “an,” and “the” designate both the singular and the plural, unless expressly stated to designate the singular only.

The term “about” and the use of ranges in general, whether or not qualified by the term about, means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term, or plus or minus 1%.

The phrase “substantially free of,” as used herein, means that the described composition (e.g., polymer matrix, etc.) comprises less than 5 wt. %, or less than 3 wt. %, or less than 1 wt. %, %, or less than 0.1 wt. %, based on the total weight of the composition at issue, of the excluded component(s). The phrase “free of” as used herein means that the described composition (e.g., polymer matrix, etc.) is formulated without adding the excluded component(s) as an intended component, although trace amounts may be present in other components or as a by-product or contaminant, such that the composition comprises at most only trace amounts of the excluded component(s).

As used herein “subject” denotes any mammal in need of treatment, including humans. For example, a subject may be suffering from or at risk of developing a condition that can be treated, inhibited, or prevented with a drug, or may be taking a drug for other purposes.

As used herein, the terms “topical” and “topically” mean application to a skin surface of a mammal, while the terms “transdermal” and “transdermally” connote passage through the skin, into systemic circulation. Thus, the compositions described herein may be applied topically to a subject to achieve topical and/or transdermal delivery of a drug or other active agent.

As used herein, the phrases “therapeutically effective amount” and “therapeutic level” mean that a dosage or plasma concentration in a subject, respectively, that provides the specific pharmacological effect for which the drug or other active agent is administered in a subject in need of such treatment. It is emphasized that a therapeutically effective amount or therapeutic level of a drug will not always be effective in treating the conditions/diseases described herein, even though such dosage is deemed to be a therapeutically effective amount by those of skill in the art. For convenience, only, exemplary dosages, drug delivery amounts, therapeutically effective amounts and therapeutic levels are provided below with reference to adult human subjects. Those skilled in the art can adjust such amounts in accordance with standard practices as needed to treat a specific subject and/or condition/disease. A “cosmetically effective amount” refers to an amount sufficient to provide a cosmetic effect when a device or composition as described herein is placed in contact with the skin.

The devices and compositions described herein are in a “flexible, finite form.” As used herein, the phrase “flexible, finite form” means a substantially solid form capable of conforming to a surface with which it comes into contact, and capable of maintaining contact to facilitate topical application. Such systems in general are known in the art and commercially available, such as transdermal drug delivery patches.

In one exemplary embodiment, the compositions in flexible, finite form are “monolithic” or “monolayer” systems, such that the active agent-containing active layer is the only layer present other than the backing layer and the release liner, if present. In other words, the polymer matrix functions as both the drug carrier and the means of affixing the system to the skin or mucosa.

In another exemplary embodiment, the compositions in flexible, finite form are multilayer systems, such that the active layer includes two or more layers, other than the backing layer and the release liner, if present. In other words, the polymer matrix functions as the drug carrier, while a separate layer provides the means of affixing the system to the skin or mucosa.

The active layer disclosed herein provides good drug release properties but also good physical properties (e.g., good adherence to skin, adherence under adverse conditions, ability to be peeled or otherwise removed without substantial trauma to the skin, retention of tack with aging, etc.).

An “active agent” can be an active pharmaceutical ingredient (API) and/or an active cosmetic ingredient. An API refers to at least one ingredient capable of treating at least one condition of the body. In some embodiments, the active agent provides a pharmacological treatment and/or care of the skin and/or mucous membranes which alleviates or eliminates a disease or disorder or reduces or eliminates one or more symptoms associated with such disease or disorder. In some embodiments, the skin and/or mucous membranes are permeable to the active agent allowing the active agent to provide a pharmacological treatment and/or care to underlying parts of the body which alleviates or eliminates a disease or disorder or reduces or eliminates one or more symptoms associated with such disease or disorder. In some embodiments, the active agent includes or consists of an active cosmetic ingredient which provides a cosmetic, non-therapeutic treatment and/or care of the skin and/or mucous membranes.

The term “skin” is understood to be the layers which comprise it, from the uppermost layer or stratum corneum to the lowermost layer or hypodermis, both inclusive. These layers are composed of different types of cells such as keratinocytes, fibroblasts, melanocytes, mast cells, neurons and/or adipocytes among others. The term “skin” also includes the scalp.

The term “polymer matrix” refers to polymeric components of the active layer and includes a thermoplastic polyurethane polymer, a pressure sensitive adhesive, optionally a poly(meth)acrylate. The term “polymer matrix” excludes active agents, such as peptides, proteins, drugs, etc., water, and non-polymeric excipients, such as solvents.

A “polymeric component”, as used herein, refers to a polymer derived from at least 10, or at least 20 monomer units (which can be the same or different) and having a molecular weight of at least 800 daltons.

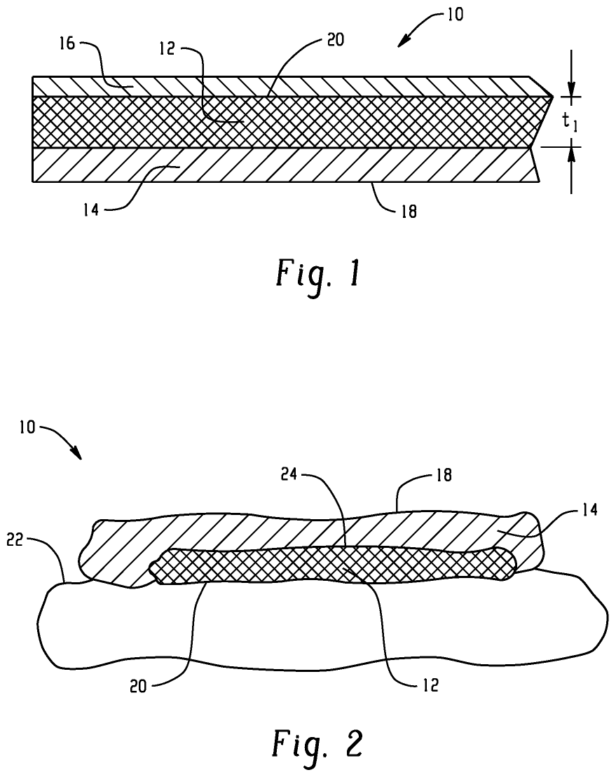

With reference to FIG. 1, a topical or transdermal delivery device 10, in the form of a patch, suitable for delivering an active agent to a skin surface is shown. The device 10 includes or consists of an active layer 12 (a monolayer) which includes the active agent, and one or both of a backing layer 14 and a removable release liner 16, which are spaced from each other by the active layer. As illustrated in FIG. 2, when in use, with the release liner removed, the backing layer 14 defines an outer surface 18 of the device 10, and the active layer 12 defines an inner surface 20 of the device which contacts a surface 22 of the skin of a wearer. The respective opposed surfaces of the backing layer and the active layer are in direct contact in a join region 24.

In the embodiments of FIGS. 1 and 2, the active layer 12, of itself, is sufficiently adhesive to attach firmly to the wearer's skin, allowing the active agent contained therein to pass from the active layer 12 onto/into the wearer's skin. Layer 12 may include a polymer matrix, which consists of polymeric materials, specifically, a thermoplastic polyurethane, a pressure sensitive adhesive, and optionally, a poly(meth)acrylate polymer. Other additives may be incorporated in the active layer, including the active agent. The active agent may be dispersed, e.g., homogeneously, throughout the active layer 12. The active layer 12 may have a coating weight of at least 2 mg/cm², or at least 5 mg/cm², or at least 10 mg/cm², or up to 50 mg/cm², or up to 30 mg/cm². The coating weight correlates to thickness and may depend on the length of time the device is to be used. Larger thicknesses may be more suitable for longer times. A thickness t₁ of layer 12 may be at least 0.1 μm or at least 1 μm, or at least 10 μm.

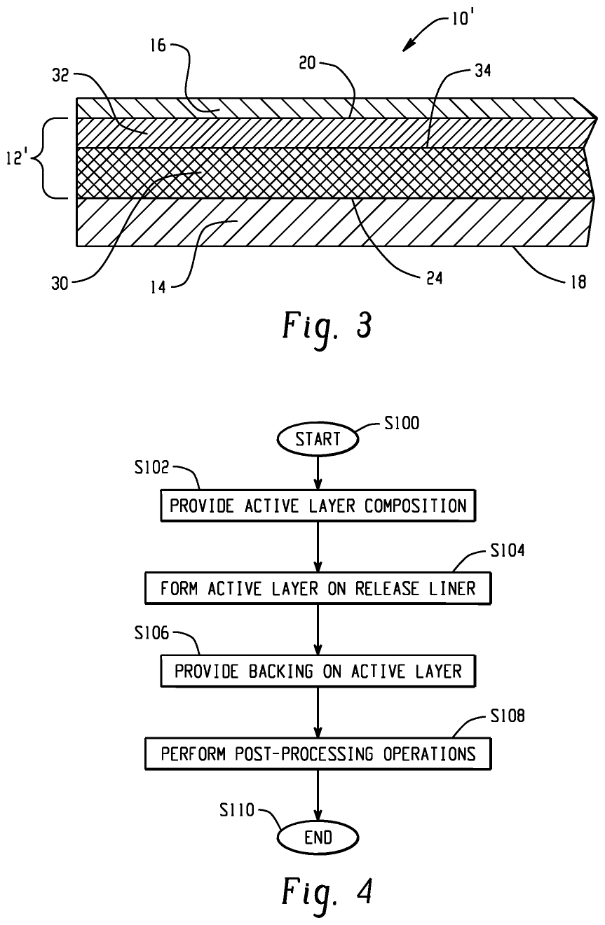

In another embodiment, shown in FIG. 3, a topical or transdermal device 10′, in the form of a patch, suitable for delivering an active agent to a skin surface is shown. Similar elements are accorded the same numerals. This embodiment partitions the components of the layer 12 of FIG. 1 into sub-layers. The active layer 12′ of FIG. 3 includes at least two sub-layers. The first sub-layer 30 includes at least one active agent. Layer 30 may be free or substantially free of pressure sensitive adhesive. The second sub-layer 32 includes an adhesive, such as a pressure sensitive adhesive, for adhering the active layer 12′ and backing layer 14 to the skin. Layer 32 may initially be free or substantially free of the at least one active agent. However, the active agent will pass through this layer to reach the skin. In this embodiment, when in use, the backing layer 14 defines an outer surface 18 of the device, and the adhesive layer 32 defines an inner surface 20 of the device which contacts a surface 22 of the skin of a wearer. Adjacent surfaces of the backing layer and sub-layer 30 make contact, directly or indirectly (e.g., through a second adhesive layer, not shown), in a first join region 24, while sub-layers 30 and 32 make contact in a second join region 34. The active layer 12′ may have a coating weight of at least 4 mg/cm², or at least 8 mg/cm², or at least 15 mg/cm², or up to 65 mg/cm², or up to 50 mg/cm². The sub-layer 30 may have a coating weight of at least 2 mg/cm², or at least 5 mg/cm², or up to 50 mg/cm², or up to 40 mg/cm². The sub-layer 32 may have a coating weight of at least 2 mg/cm², or at least 5 mg/cm², or up to 15 mg/cm², or up to 12 mg/cm². A thickness t₂ of layer IT may be at least 0.1 μm or at least 1 μm, or at least 10 μm, where t₂=t₃+t₄.

In one embodiment, sub-layer 30 includes a polymer matrix, which includes a thermoplastic polyurethane, and optionally, a poly(meth)acrylate polymer. Other polymeric materials and/or other additives may be incorporated in the active layer. The active agent may be dispersed, e.g., homogeneously, throughout the sub-layer 30.

A composition (I) for forming the active layer 12 of the device of FIGS. 1 and 2 may include a polymer matrix including a thermoplastic polyurethane polymer, a pressure sensitive adhesive, and optionally, a poly(meth)acrylate polymer. The composition (I) further includes a therapeutically effective amount of an active pharmaceutical ingredient dispersed in the polymer matrix. The composition (I) optionally includes one or more other (non-polymeric) additives, e.g., acting as excipients, fillers, or the like.

A composition (II) for forming the sub-layer 30 of the active layer 12′ of the device of FIG. 3 may include a polymer matrix including a thermoplastic polyurethane polymer, and optionally, a poly(meth)acrylate polymer. The composition (II) further includes a therapeutically effective amount of an active pharmaceutical ingredient dispersed in the polymer matrix. The composition (II) optionally includes one or more other (non-polymeric) additives.

A composition (III) for forming the sub-layer 32 of the active layer 12′ of the device of FIG. 3 may include a pressure sensitive adhesive and optionally a TPU, such as one of the TPU's described herein. In one embodiment, a ratio of TPU:PSA in layer 32 is at least 1:10, such as at least 1:5, or at least 1:2, or up to 2:1, such as about 1:1. The sub-layer 32 optionally includes one or more other (non-polymeric) additives, as described below. With time, those compounds which permeate from sub-layer 30 to the skin will reach equilibrium in sub layer 32. As will be appreciated, the sub-layer 32 is adhered to the layer 30.

The thermoplastic polyurethane polymer employed in the active layer 12 (or sub-layer 30) may be the reaction product of:
(i) at least one of:
   a) a first polyether polyol A, having a molecular weight of at least 3000 daltons, or at least 4000 daltons, and
   b) a second polyether polyol B, having a molecular weight of no more than 2500 daltons, or no more than 2000 daltons;
(ii) at least one of:
   c) a third polyol C having a molecular weight of up to 800 daltons, and
   d) a chain extender D;
(iii) a polyisocyanate; and
(iv) optionally, a catalyst.

In one embodiment, in the composition forming the active layer described herein, Polyol A is present and has a molecular weight of at least 4000 daltons; Polyol B is present, and is a polyether polyol having a molecular weight of no more than 2500 daltons, such as no more than 2000 daltons; a ratio of the molecular weight of Polyol A to the Polyol B is at least 1.4:1. The chain extender D may have a molecular weight of up to 300 daltons.

In another embodiment, in the composition forming the active layer described herein, a ratio by weight of Polyol B to Polyol A is at least 1:1 and Polyol B has a molecular weight of no more than 2500 daltons. The chain extender D may have a molecular weight of up to 300 daltons.

In another embodiment, in the composition forming the active layer described herein, polyol A is absent and polyol B has a molecular weight of no more than 2500 daltons. The chain extender D may have a molecular weight of up to 300 daltons.

In one specific embodiment, a transdermal drug delivery device 10 includes:

1. a backing layer 14;

2. a combined adhesive and drug-containing active layer 12, 12′ comprising:
(A) a therapeutically effective amount of an active pharmaceutical ingredient;
(B) a polymer matrix including:
   (1) 20 to 70 wt. % of a thermoplastic polyurethane polymer, wherein the thermoplastic polyurethane polymer comprises the reaction product of:
      (i) at least one of:
         a) a first polyol A, which is a polyether polyol having a molecular weight of at least 3000 daltons, or at least 4000 daltons, and
         b) a second polyol B, having a molecular weight of no more than 2500 daltons, or no more than 2000 daltons;
      (ii) at least one of:
         c) a third polyol C having a molecular weight of up to 800 daltons, and
         d) a chain extender D;
      (iii) a polyisocyanate; and
         (iv) optionally, a catalyst;
   (2) 30 to 80 wt. % of a pressure sensitive adhesive;
   (3) optionally, at least 2 wt. % of a poly(meth)acrylate polymer;
and

3. optionally, a removable release liner 16;

wherein the combined adhesive and drug-containing active layer 12, 12′ has a first surface 24 and a second surface 20, and the first surface is directly affixed to a surface of the backing layer; and

wherein the removable release liner, where present, is directly affixed to the second surface of the active layer.

### Components of the Active Layer

In the embodiments described herein, the active layer 12, 12′ includes a therapeutically effective amount of an active pharmaceutical ingredient, a thermoplastic polyurethane polymer, a pressure sensitive adhesive, optionally, a poly(meth)acrylate polymer, and optionally one or more other additives. The polymeric components (including the thermoplastic polyurethane polymer, pressure sensitive adhesive and poly(meth)acrylate polymer, where present) may be in the form of a polymer matrix. Alternatively, at least some of the pressure sensitive adhesive may from a separate layer.

In one embodiment, the thermoplastic polyurethane polymer, pressure sensitive adhesive, and poly(meth)acrylate polymer, where present, constitute at least 80 wt. %, or at least 90 wt. %, or up to 99.8 wt. % of the active layer.

### A. Polymer Matrix

In the embodiment of FIGS. 1-3, the polymer matrix (i.e., solely the polymer components of the active layer 12, 12′) may be at least 20 wt. % of the active layer 12, 12′, such as at least 30 wt. %, or at least 40 wt. %, or at least 50 wt. % and in some embodiments up to 99.9 wt. %, or up to 90 wt. % of the active layer 12, 12′.

In specific embodiments, the polymer matrix may be at least 30 wt. % of the active layer 12 of FIG. 1, such as at least 40 wt. %, or at least 50 wt. %, or at least 60 wt. % and in some embodiments, up to 90 wt. %, or up to 80 wt. %, or up to 70 wt. % of the active layer 12. The balance of the active layer 12 may comprise or consist of the active agent and any non-polymeric additives present.

In the embodiment of FIG. 3, the polymer matrix may be at least 20 wt. of the active layer 12′, such as at least 30 wt. %, or at least 40 wt. %, or at least 50 wt. % and in some embodiments up to 70 wt. %, or up to 60 wt. % of the active layer 12′. The balance of the active layer 12′ of FIG. 3 may comprise or consist of the active agent, the pressure sensitive adhesive, and any non-polymeric additives present.

### 1. Thermoplastic Polyurethane Polymer

The exemplary thermoplastic polyurethane polymer (or TPU) includes a hard segment, generally made up of the polyisocyanate and chain extender E, a soft segment, generally made up of the polyols A and/or B, and optionally an intermediate segment, generally where the polyisocyanate joins polyols A and B. The hard segment has a hardness greater than the soft segment.

The TPU may be present in the composition forming the active layer 12 (or forming sub-layer 30) at 20 to 70 wt. %, or from 45 to 55 wt. %, or about 50 wt. %, or at least 20 wt. %, or at least 30 wt. %, or at least 40 wt. % or at least 45 wt. %, or at least 50 wt. %, or at least 60 wt. %, or up to 80 wt. %, or up to 70 wt. %, or up to 55 wt. %, or up to 50 wt. %.

In the embodiment of FIG. 1, for example, the TPU may be at least 20 wt. % of the polymer matrix in layer 12, such as at least 30 wt. %, or at least 40 wt. %, or at least 50 wt. %, or at least 60 wt. %, and in some embodiments up to 80 wt. %, or up to 70 wt. % of the polymer matrix.

In the embodiment of FIG. 3, the thermoplastic polyurethane polymer may be at least 30 wt. % of the polymer matrix in sub-layer 30, such as at least 40 wt. %, or at least 50 wt. %, or at least 60 wt. %, or at least 70 wt. %, and in some embodiments up to 100 wt. %, or up to 98 wt. %, or up to 90 wt. %, or up to 80 wt. of the polymer matrix.

All molecular weights mentioned herein are weight average molecular weights and are expressed in daltons, unless otherwise noted.

The exemplary TPU is the reaction product of a combination of reactants selected from:

a. Polyol A

Polyol A is a polyether polyol and may have an average molecular weight of at least 3000 daltons, or at least 4000, or at least 6000 daltons, and in some embodiments, up to 15,000 daltons, or up to 12,000, or up to 10,000 daltons.

Polyol A may be of the general form HO—(R₁(R₂)O)ₙ—H, where: R₁ is selected from C₂-C₄ alkyl groups and mixtures thereof, such as CH₂CH or CH₂CH₂CH, R₂ is selected from H and C₁-C₂ alkyl groups and mixtures thereof, and n is an integer which represents the average number of ether units by weight in each polyol, and where n is at least 20.

The value of n for Polyol A may be at least 60 or at least 100 or at least 150 and may be up to 500 or up to 240.

b. Polyol B

In one embodiment, a first Polyol B (referred to for convenience as Polyol B1) includes or consists of at least one polyether polyol which has an average molecular weight of no more than 2500 daltons, or no more than 2000, or no more than 1600 daltons, or no more than 1200 daltons, and in some embodiments, at least 800 daltons, or at least 1000 daltons, or at least 1200 daltons. Polyol B1, in this embodiment, may be of the general form HO—(R₁(R₂)O)ₙ—H, where: R₁ is selected from C₂-C₄ alkyl groups and mixtures thereof, such as CH₂CH or CH₂CH₂CH, R₂ is selected from H and C₁-C₂ alkyl groups and mixtures thereof, and n is an integer which represents the average number of ether units by weight in each polyol. In one embodiment, at least a portion of the R₂ groups in polyol B1 are not H. In one embodiment Polyol B1 is a copolymer in which some R₂ groups are H and some R₂ groups are C₁-C₂ alkyl groups. The value of n is dependent upon R₁ and R₂ to provide a total molecular weight within the desired range. The value of n for Polyol B1, in this embodiment, may be at least 20, or at least 25, or at least 30, and may be up to 50 or up to 45. Examples of polyols suitable as polyol B1, in this embodiment, include copolymers of ethylene oxide and propylene oxide, such as PTMEG, and mixtures thereof. List here example HO—(R₁(R₂)O)ₙ—H polyols for B1.

In another embodiment, Polyol B (referred to for convenience as Polyol B2) includes or consists of at least one polyether polyol having a molecular weight of at least 1200 daltons and/or up to 2500 daltons. Polyol B2 may be of the general form HO—(R₁O)ₙ—H, where: R₁ is selected from C₂-C₄ alkyl groups and mixtures thereof, such as CH₂CH₂ or CH₂CH₂CH₂, and n is an integer which represents the average number of ether units by weight in each polyol, and where n is at least 20. The value of n for Polyol B2, in this embodiment, is dependent upon the choice of R₁ in order to provide a total molecular weight in the desired range.

Examples of polyols suitable as Polyol B2, in this embodiment, include polyalkylene-ether glycols, such as polyethylene glycol (PEG), polytrimethylene glycol, poly(tetramethylene ether) glycol (PTMG), poly(trimethylene-ethylene ether) glycol (a copolymer of polytrimethylene glycol and ethylene glycol) (PTEMG), and mixtures thereof.

In one embodiment, Polyol B2 is a blend of polyethylene glycol (e.g., of molecular weight 1300-1600 daltons) and poly(tetramethylene ether) glycol (PTMG) (e.g., of molecular weight 1300-1600 daltons).

In one embodiment, Polyol A is optional and both Polyols B1 and B2 are present.

In one embodiment, Polyol A is present and at least one of Polyols B1 and B2 is present.

In one embodiment Polyol A is absent and at least one of Polyols B1 and B2 is present.

In one embodiment, Polyol B1 is present and includes at least some C₁-C₂ alkyl groups as R₂.

c. Polyol C

A molecular weight of Polyol C may be at least 200, or at least 250, or at least 300 daltons and may be up to 800 or up to 500, or up to 400 daltons. Polyol C may be a polyether polyol as for Polyols A and B. The value of n is dependent upon the selection of R₁ and R₂, in order to provide a total molecular weight in the range specified. For example, n is at least 3 or at least 5, or at least 7, and may be up to 20 or up to 12.

Suitable hydroxyl terminated polyether polyols which are useful as Polyols A, B and C are derived from a diol or polyol having a total of, for example, from 2 to 15 carbon atoms, such as an alkyl diol or glycol, which is reacted with an ether comprising an alkylene oxide of, for example, from 2 to 6 carbon atoms, such as ethylene oxide, propylene oxide or a mixture thereof, or through an acid catalyzed polycondensation reaction. For example, polyether polyols can be produced by first reacting propylene glycol with propylene oxide followed by subsequent reaction with ethylene oxide.

Example polyether polyols include poly(ethylene glycol) formed by reaction of ethylene oxide with ethylene glycol, poly(propylene glycol) formed by reaction of propylene oxide with propylene glycol, a copolymer of ethylene oxide and a C₃ to C₁₅ alkylene oxide, in particular, a C₃ to C₆ alkylene oxide, such as poly(propylene glycol), e.g., a copolymer polyol comprising poly(1,2 propylene glycol) and poly(ethylene oxide) segments, poly(tetramethylene ether glycol) formed by reaction of water with tetrahydrofuran, which can also be described as polymerized tetrahydrofuran, and which is referred to herein as PTMG. Suitable polyether polyols also include polyamide adducts of an alkylene oxide and can include, for example, ethylenediamine adduct formed by reaction of ethylenediamine with propylene oxide, diethylenetriamine formed by reaction of diethylenetriamine with propylene oxide, and similar polyamide type polyether polyols.

Copolyethers can also be utilized in the described compositions. Typical copolyethers include the reaction product of THF and ethylene oxide or THF and propylene oxide. These are available from BASF as PolyTHF® B, a block copolymer, and PolyTHF® R, a random copolymer.

d. Chain Extender D

The exemplary TPU composition may be made using a chain extender (chain lengthening agent) D. The chain extender D can be used in addition to or in place of Polyol C. The chain extender may also be a polyol, such as a C₁-C₂₀ or C₁-C₁₂, or C₂-C₁₀ alkyl and/or aryl polyol. A molecular weight of the chain extender may be up to 300 daltons, or up to 250 daltons, or up to 200 daltons.

Chain extenders include diols, diamines, and combinations thereof. The chain extender may have a molecular weight of up to 500 daltons or up to 300 daltons, such as at least 46 daltons.

One or more short chain polyols having from 2 to 20, or 2 to 12, or 2 to 10 or 2-8 carbon atoms may be used as chain extenders in the polyurethane forming composition to increase the molecular weight of the polyurethane. Examples of chain extenders include lower aliphatic polyols and short chain aromatic glycols having molecular weights of less than 500 or less than 300. Suitable chain extenders include organic diols (including glycols) having a total of from 2 to 20 carbon atoms such as alkane diols, cycloaliphatic diols, alkylaryl diols, and the like. Exemplary alkane diols include ethylene glycol, diethylene glycol, 1,3 propanediol, 1,3 butanediol, 1,4 butanediol, (BDO), 1,3-butanediol, 1,5-pentanediol, 2,2-dimethyl-1,3-propanediol, propylene glycol, dipropylene glycol, 1,6-hexanediol, heptanediol, nonanediol, 1,10-decanediol, dodecanediol, tripropylene glycol, triethylene glycol, and 3-methyl-1,5-pentanediol. Examples of suitable cycloaliphatic diols include 1,2-cyclopentanediol, and 1,4-cyclohexanedimethanol (CHDM). Examples of suitable aryl and alkylaryl diols include hydroquinone bis(2-hydroxyethyl)ether (HQEE), 1,2-dihydroxybenzene, 1,3-dihydroxybenzene, 1,4-dihydroxybenzene, 1,2,3-trihydroxybenzene, 1,2-bis(hydroxymethyl)benzene, 1,4-bis(hydroxymethyl)benzene, 1,3-bis(2-hydroxyethyl)benzene, 1,2-bis(2-hydroxyethoxy)benzene, 1,4-bis(2-hydroxyethoxy)benzene, bisethoxy biphenol, 2,2-bis(4-hydroxyphenyl)propane (i.e., bisphenol A), bisphenol A ethoxylates, bisphenol F ethoxylates, 4,4′-isopropylidenediphenol, 2,2-bis[4-(2-hydroxyethoxy)phenyl]propane (HEPP), and mixtures thereof and the like.

Mixtures of one or more of the above chain extenders can also be utilized.

Chain extenders with functionality greater than 2 may be used so long as the resulting TPU retains its thermoplasticity. Examples of such chain extenders include trimethylolpropane (TMP), glycerin and pentraerythritol. Generally, the addition of such chain extenders should not exceed 10% relative to the weight of the difunctional chain extenders.

In one embodiment, the chain extender is selected from 1,4-butanediol and 1,10-decanediol.

Chain extenders can also be based on diamines. Exemplary diamines may have molecular weights of less than 500, and include, for example, as ethylenediamine, diethylenediamine, tetramethylenediamine, hexamethylenediamine, diethylenetriamine triethylenetetramine, tetraethylenepentamine, pentaethylenehexamine, piperazine, morpholine, substituted morpholine, piperidine, substituted piperidine, 2-amino-1-ethylpiperazine hydrazine, 1,4 cyclohexanediamine, and mixtures thereof. Alkanolamines, such as ethanolamine, diethanolamine, and triethanolamine, may also be used. Further examples of chain lengthening agents include aniline, and the like. The molar amount or ratio of the total hydroxyl groups of the one or more chain extenders utilized to the total hydroxyl groups of Polyols A, B, and C (where present) may be from 0.1 to 5.0, or from 0.2 to 4.0, or from 0.4 to 2.5.

e. Polyisocyanate

The polyisocyanate includes one or more polyisocyanates. Suitable polyisocyanates include aromatic diisocyanates, aliphatic diisocyanates, and mixtures thereof.

In order to form relatively long linear polyurethane chains, di-functional or polyfunctional isocyanates are utilized. In one embodiment, one or more diisocyanates are utilized. The polyisocyanates useful herein generally have a formula R(NCO)n, where n is at least 2. R can be an aromatic, a cycloaliphatic, an aliphatic, or combinations thereof having from 2 to 20 carbon atoms.

Examples of aromatic diisocyanates include 4,4′-diphenylmethane diisocyanate (MDI), m-xylene diisocyanate (XDI), phenylene 1,4 diisocyanate, naphthalene 1,5 diisocyanate, 2,4 toluene diisocyanate (2,4-TDI), 2,6-toluene diisocyanate (2,6-TDI), 1,4-phenylene diisocyanate, 1,3-phenylene diisocyanate, 4,4-diphenylene diisocyanate, 3,3-dimethoxy-4,4-biphenylene diisocyanate, 3,3′-dimethyl-4,4′-biphenylene diisocyanate (TODD, 4,4-diisocyanate diphenyl ether, 2,4-naphthalene diisocyanate, 1,5-naphthalene diisocyanate, 1,3-xylene diisocyanate, 1,4-xylene diisocyanate.

Examples of aliphatic diisocyanates include 1,4-butane diisocyanate (BDI), tetramethylene diisocyanate, hexamethylene diisocyanate (HDI), 1,4-cyclohexyl diisocyanate (CHDI), decane-1,10-diisocyanate, lysine diisocyanate (LDI), dicyclohexylmethane-4,4′-diisocyanate (H12MDI), 1,3-diisocyanate methylcyclohexane, 1,4-diisocyanate methylcyclohexane, 4,4-diisocyanate dicyclohexane, 4,4-diisocyanate dicyclohexylmethane, isophorone diisocyanate, and isomers, dimers, trimers and mixtures thereof.

In one embodiment, the isocyanate is selected from cycloaliphatic and aromatic diisocyanates. In one specific embodiment, the isocyanate is selected from MDI and H12MDI.

Suitable polyisocyanates are commercially available, for example, from Bayer Corporation of Pittsburgh, Pa., The BASF Corporation of Parsippany, N.J., The Dow Chemical Company of Midland, Mich., and Huntsman Chemical of Utah.

f. Catalyst

One or more polymerization catalysts may be present during the polymerization reaction. Generally, any conventional catalyst can be utilized to react the diisocyanate with the polyol intermediates and/or the chain extender. Examples of suitable catalysts which in particular accelerate the reaction between the NCO groups of the diisocyanates and the hydroxy groups of the polyols and chain extenders include organic tin compounds such as dibutyltin diacetate, dibutyltin dilaurate (DBTL), dioctyltin dilaurate (DOTDL), and dibutyltin bis(ethoxybutyl-3-mercaptopropionate); titanic acid; organic titanium compounds such as tetraisopropyl titanate, tetra n butyl titanate, polyhydroxytitanium stearate and titanium acetylacetonate; tertiary amines such as triethylene diamine, triethylamine N-methylmorpholine, N,N,N′,N′-tetramethylethylenediamine, N,N,N′,N′-tetramethylhexamethylene diamine, triethylamine, N,N′-dimethylpiperazine, N,N-dimethylaminoethanol, dimethylcyclohexylamine, diazabicyclo[2.2.2]octane; and mixtures of two or more thereof. The total amount of the catalyst used may be from 0.0001 to 0.1 part by weight per 100 parts by weight of the polyols A, B, and C.

### Example Thermoplastic Polyurethanes

The thermoplastic polyurethanes described herein have a soft segment and a hard segment. The soft segment may be derived from at least one of polyols A and B. The hard segment may be derived from a polyisocyanate (or a diisocyanate), and at least one of a third polyol (Polyol C) and a chain extender D.

i. TPU1

A TPU (denoted TPU1) includes a soft segment which is derived from a first polyether polyol (Polyol A) and a second polyether polyol B (e.g., at least Polyol B1), which is of lower molecular weight than Polyol A. A ratio of the value of n for polyol A to the value of n for polyol B may be at least 1.5:1 or at least 2:1, or at least 3:1, or at least 4:1, and may be up to 20:1 or up to 10:1.

A ratio of the molecular weight of Polyol A to polyol B may be at least 1.4:1 or at least 2:1 and may be up to 15:1 or up to 10:1, or up to 8:1. The soft segment thus has a bimodal distribution of average length of the polyether segments.

A molar ratio of Polyol B to Polyol A in the reaction mixture for forming TPU1 may be at least 5:1 or at least 8:1 such as up to 20:1. A weight ratio of Polyol B1 to Polyol A in forming the thermoplastic polyurethane may be at least from 1:1 to 8:1, such as at least 1.5:1, or at least 8:1.

An example thermoplastic polyurethane TPU1 is the reaction product of: 20-30 wt. % Polyol A, 40-70 wt. % Polyol B (e.g., B2), 5-20 wt. % polyisocyanate, 0.2-5 wt. % Polyol C, 0-5 wt. % chain extender, such as at least 0.2 wt. %, optionally, one or more additional polymer forming components, and optionally up to 2 wt. % of a catalyst (totaling 100 wt. %). In combination, Polyols A and B may comprise at least 60 wt. %, or at least 70 wt. % of the total weight of polymer-forming components in the reaction mixture. In this embodiment, polyol B may have a molecular weight of no more than 2500 daltons.

TPU1 may have a weight average molecular weight of from 50,000 to 1,000,000 daltons, or from 75,000 to 500,000, or from 100,000 to 300,000 daltons.

ii. TPU2

A TPU (denoted TPU2) is as described for TPU1 but includes Polyol A but not Polyol B (or only a minor amount, such as up to 5 wt. %). It may further include polyol C.

An example thermoplastic polyurethane TPU2 is the reaction product of: 70-90 wt. % Polyol A, 5-20 wt. % polyisocyanate, 0-15 wt. % Polyol C, 0-10 wt. chain extender, optionally, one or more additional polymer forming components, and optionally up to 2 wt. % of a catalyst (totaling 100 wt. %).

iii. TPU3

A TPU (TPU3) is as described for TPU1 but includes Polyol B but not Polyol A (or no more than 5 wt. % Polyol A). TPU3 includes a soft segment which is derived from at least one polyether Polyol B, such as at least two polyether Polyols B which are of similar molecular weight but have different R₂ groups, such as B1, where R₂ may be a mixture of H and C₁-C₂ alkyl groups, and B2.

An example thermoplastic polyurethane TPU3 is the reaction product of: 30-90 wt. % Polyol B, 5-50 wt. % polyisocyanate, 0-5 wt. % Polyol C, 0-20 wt. % chain extender, optionally, one or more additional polymer forming components, and optionally up to 2 wt. % of a catalyst (totaling 100 wt. %).

TPU3 may have a weight average molecular weight of from 50,000 to 1,000,000 daltons, or from 75,000 to 500,000, or from 100,000 to 300,000 daltons.

Blends of two or more of the thermoplastic polyurethanes TPU1, TPU2, and TPU3 are also contemplated. A weight ratio of TPU1:TPU2 and/or TPU3 may be at least 1:2. In one embodiment, the ratio is at least 1:1.5, or at least 1:1, or at least 2:1, and may be up to 100:1, or higher, such as up to 20:1, or up to 10:1.

The exemplary thermoplastic polyurethane is optionally blended in with one or more additional polymers, such as those used as the pressure sensitive adhesive and/or poly(meth)acrylate polymer, as described below. Additional polymers suitable for use herein include thermoplastic polyurethanes, amine-based polymers, and cross-linked poly(acrylic acid) polymers.

2. The Pressure Sensitive Adhesive

The active layer 12, 12′ includes a pressure sensitive adhesive (PSA), which may be part of the polymer matrix (FIG. 1) and/or form a separate sub-layer 32 (FIG. 3). The pressure sensitive adhesive may be at least 20 wt. %, or at least 30 wt. % of the active layer 12, 12′ and may be up to 80 wt. % or up to 70 wt. % of the active layer 12, 12′. In the embodiment of FIG. 1, the pressure sensitive adhesive may be at least 20 wt. %, or at least 30 wt. % of the active layer 12, and in some embodiments, up to 80 wt. %, or up to 70 wt. %, or from 30 to 80 wt. %. In some embodiments, the PSA is from 34.5 to 76.6 wt. %, or from 45 to 55, or about 50 wt. %, or from 30, 34.5, 45 or 50 wt. % up to 34.5, 45, 50, 55, 76.6, or 80 wt. % of layer 12.

In the embodiment of FIG. 3, the pressure sensitive adhesive may be at least 80 wt. %, or at least 90 wt. % of the sub-layer 32, and in some embodiments, up to 100 wt. %.

In other embodiments, the PSA may present at from 27, 30, or 53 wt. %, up to 30, 53, or 59 wt. % of layer 12′.

A material is considered an “adhesive” if it has the properties of adhesiveness, per se. Other materials, including certain polymers, can function as an adhesive or bioadhesive by the addition of tackifiers, plasticizers, crosslinking agents or other excipients. Thus, in some embodiments, the pressure-sensitive adhesive component described here may optionally comprise tackifiers, plasticizers, crosslinking agents or other additives known in the art.

As used herein, the term “pressure-sensitive adhesive” refers to a viscoelastic material which adheres instantaneously to most substrates with the application of very slight pressure and remains permanently tacky. As noted above, an adhesive is a pressure-sensitive adhesive if it has the properties of a pressure-sensitive adhesive per se. Other materials, including certain polymers, may function as a pressure-sensitive adhesive by admixture with tackifiers, plasticizers or other additives. The term pressure-sensitive adhesive also includes mixtures of different adhesives.

In some embodiments, the polymer matrix acts as a pressure-sensitive adhesive at room temperature and exhibits desirable physical properties, such as good adherence to skin, ability to be peeled or otherwise removed without substantial trauma to the skin, retention of tack with aging, etc. In some embodiments, the pressure sensitive adhesive has a glass transition temperature (Tg), measured using a differential scanning calorimeter, of between −70° C. and 0° C. and when blended with the other ingredients of the polymer matrix, the matrix may exhibit multiple transitions reflecting the block nature of TPU or a single Tg between −70° C. and 5° C.

In various embodiments, the pressure sensitive adhesive comprises one or more acrylic-based polymers, silicone-based polymers, rubbers, gums, polyisobutylenes, polyvinyl ethers, polyurethanes, styrene block copolymers, styrene-butadiene polymers, polyether block amide copolymers, ethylene/vinyl acetate copolymers, vinyl acetate-based adhesives, bioadhesives, or any combination thereof. In some embodiments, the pressure sensitive adhesive comprises one or more acrylic based adhesives, one or more silicone-based adhesives, or any combination thereof. In some embodiments, the pressure sensitive adhesive comprises one or more acrylic based adhesives. In some embodiments, the pressure sensitive adhesive comprises one or more silicone-based adhesives.

Example PSAs may be formed by copolymerization of acrylic monomers and derivatives thereof. The resulting polymer has a saturated hydrocarbon backbone and an ester side chain. A sticky acrylic pressure-sensitive polymer is obtainable by modifying the co-monomers and side chain groups. The monomers commonly used include low Tg polymer forming soft monomers, such as ethyl acrylate, 2-ethylhexyl acrylate, isooctyl acrylate, and butyl acrylate, 2-ethylbutyl acrylate, decyl acrylate, dodecyl acrylate and vinyl acetate for improving the adhesion of the pressure-sensitive polymers, hard monomers, such as vinyl acetate, methyl acrylate, styrene, acrylonitrile, C₁-C₁₀ alkyl methacrylates, e.g., methyl methacrylate, ethyl methacrylate and n-butyl methacrylate, for improving the cohesion of the pressure-sensitive polymers, and functional monomers such as (meth)acrylic acid, (meth)acrylamide, β-hydroxyethyl (meth)acrylate, β-hydroxypropyl (meth)acrylate, glycidyl (meth)acrylate, N-hydroxylmethylacrylamide, divinylbenzene, maleic acid, and maleic anhydride for forming chemical cross-links (see, e.g., Yukun Yang, “Pressure-Sensitive Adhesives,” Science Press, pp. 149-150, June 1994).

Suitable pressure sensitive adhesives for use herein include acrylic polymers, such as one or more pressure-sensitive adhesive acrylic polymers.

In some embodiments, the pressure sensitive adhesive comprises one or more non-acid functional acrylic polymers. Non-acid functional acrylic polymers include those formed from acrylic esters copolymerized with other monomers that do not include acid-functional groups. Non-acid functional acrylic polymers include homopolymers, copolymers, terpolymers, etc., of acrylate esters and methacrylate esters or acrylamides and methacrylamides and vinyl acetate. As used herein, “non-acid functional acrylic polymer” includes polymers that include monomers that have one or more amide groups.

In some embodiments, the pressure sensitive adhesive may include one or more acrylic polymers that include functional groups. Example acrylic-based polymers having functional groups are copolymers or terpolymers which contain, in addition to the nonfunctional monomer units described above, further monomer units having free functional groups. The monomers can be monofunctional or polyfunctional. These functional groups include carboxyl groups, hydroxy groups, amino groups, amido groups, epoxy groups, etc. Functional monomers that are copolymerizable alkyl acrylates or methacrylates include acrylic acid, methacrylic acid, maleic acid, maleic anhydride, hydroxyethyl acrylate, hydroxypropyl acrylate, acrylamide, dimethylacrylamide, acrylonitrile, dimethyl aminoethyl acrylate, dimethyl aminoethyl methacrylate, tert-butyl aminoethyl acrylate, tert-butyl aminoethyl methacrylate, methoxyethyl acrylate and methoxyethyl methacrylate and other monomers having at least one unsaturated double bond which participates in copolymerization reaction in one molecule and a functional group on its side chain such as a carboxyl group, a hydroxyl group, a sulfoxyl group, an amino group, an amino group and an alkoxyl, etc. Further examples include monomers with at least one of the following moieties: alkylene, hydroxy-substituted alkylene, carboxylic acid-substituted alkylene, vinyl alkanoate, vinylpyrrolidone, vinylpyridine, vinylpyrazine, vinylpyrrole, vinylimidazole, vinylcaprolactam, vinyloxazole, vinyl acetate, vinyl propionate, and vinyl morpholine. Exemplary carboxyl functional monomers include acrylic acid, methacrylic acid, itaconic acid, maleic acid, and crotonic acid. Exemplary hydroxy functional monomers include 2-hydroxyethyl methacrylate, 2-hydroxyethyl acrylate, hydroxymethyl acrylate, hydroxymethyl methacrylate, hydroxyethyl acrylate, hydroxyethyl methacrylate, hydroxypropyl acrylate, hydroxypropyl methacrylate, hydroxybutyl acrylate, hydroxybutyl methacrylate, hydroxyamyl acrylate, hydroxyamyl methacrylate, hydroxyhexyl acrylate, hydroxyhexyl methacrylate.

Thus, the pressure-sensitive adhesive may include one or more non-acid functional acrylic polymers and, optionally, one or more functional acrylic polymers. When the acrylic polymer component includes both functional and non-acid functional acrylic polymers, the polymers can be present in any ratio that results in a product with satisfactory physical and pharmacokinetic properties. For example, the pressure-sensitive adhesive can include from 0-100% of non-acid functional acrylic polymer(s) and from 100-0% of functional acrylic polymer(s), based on the total dry weight of the pressure-sensitive adhesive, including 10 to 90%, 15 to 85%, 20 to 80%, 25 to 75%, 33 to 66%, and 50% of non acid-functional acrylic polymer(s), and the balance being the functional acrylic polymer(s).

Suitable acrylic pressure sensitive adhesive polymers which are commercially available include those sold by Henkel (Dusseldorf, Germany), under the Duro-Take brand, such as Duro-Tak®87-2516, Duro-Take 87-900A, Duro-Take 87-9088, Duro-Take 87-4098, and Duro-Take 87-9900, and those sold by Monsanto (St. Louis, Mo.) under the Gelva® Multipolymer Solution brand, such as Gelva® 3087 and Gelva® 3235. Such acrylic adhesives may have a hydroxyl or carboxyl functional group, e.g., Duro-Tak®87-2516, or may have no functional group, such as Duro-Take 87-900A and Duro-Take 87-4098. Note that the Duro-Take 87-series are also sold as Duro-Take 387-.

In one embodiment, the pressure sensitive adhesive includes at least one vinyl acetate acrylic pressure sensitive adhesive Suitable examples include Duro-Tak 387-2516, Duro-Take 737NA, Duro-Take 387-2051, and Duro-Take 788NA, available from Henkel, or any combination thereof.

Some example acrylic PSAs may be copolymerized from butyl acrylate, isooctyl acrylate, vinyl acetate, acrylamide, and α-methacrylic acid monomers. For example, the copolymer (A) of Example 2 in Table 1 on page 21 of CN1640500A (Beijing Kangbeide Pharmaceuticals Co., Ltd), PAS-10-K, is copolymerized from 33.1% butyl acrylate, 40.9% isooctyl acrylate, 21.0% of vinyl acetate, 3.8% of acrylamide and 1.2% of α-methacrylic acid.

In one embodiment, the pressure sensitive adhesive includes or consists of a methacrylate polymer and/or an N,N-substituted derivative thereof, which may be prepared from a methacrylamide monomer or a N,N-substituted derivative thereof.

In one embodiment, the pressure sensitive adhesive includes at least one siloxane pressure-sensitive adhesive. Suitable siloxane PSAs typically include a linear siloxane polymer and an MQ resin composed of triorganosiloxane (M) units (i.e., R₃SiO_{1/2} units, in which R denotes a monovalent organic group) and silicate (Q) units (i.e., SiO_{4/2} units). Both standard and amine compatible silicone adhesives are available. In addition to the above two ingredients, silicone PSA compositions may be provided with a crosslinking agent. Due to the high viscosity imparted by the polymer component, such PSA compositions may be dispersed in an organic solvent. Siloxane-based PSAs are described, for example, in U.S. Pat. Nos. 5,366,809, 7,687,591, and 8,206,831, for example. Silicone adhesives are available from Dow Corning under the trade name Bio-PSA Silicone adhesives.

In one embodiment, the PSA may include poly(N-vinyl pyrrolidone) (PVP). The PVP may be at least 0.1 wt. %, or at least 1 wt. %, or up to 5 wt. %, or up to 3 wt. %, or up to 2 wt. % of the PSA component. The PVP present impacts the mechanical properties and can change the dispersion state of the drug in the composition.

The PSA may include a crosslinking agent to achieve metal based or ionic crosslinking. Example crosslinking agents include Ti-acetylacetonate, Al-acetylacetonate, polybutyl-titanate, and mixtures thereof. Examples include DURO-TAK® 387-2516 (or DURO-TAK® 87-2516). The crosslinking agent may be incorporated in the PSA when formed and/or may be added separately to provide a concentration suitable for achieving the cohesion and adhesion of the active later and the release profile for the active agent.

In one specific embodiment, the PSA includes 2-ethylhexylacrylate, vinyl acetate, hydroxyethyl acrylate, and polybutyl titanate. In another embodiment, the PSA includes 2-ethylhexylacrylate, hydroxyethylacrylate, acrylic acid, and vinyl acetate.

A ratio by weight of the TPU:PSA in the active layer 12, 12′ may be at least 1:4, such as from 1:3 to 3:1, or at least 1:1.2, or at least 1:1, or at least 2:1.

Other suitable acrylic polymers in addition to those mentioned above are known in the art. See, e.g., the non-acid functional acrylic polymers described in Satas, “Acrylic Adhesives, HANDBOOK OF PRESSURE-SENSITIVE ADHESIVE TECHNOLOGY, 2nd ed., pp. 396-456 (D. Satas, ed.), Van Nostrand Reinhold, N.Y. (1989); “Acrylic and Methacrylic Ester Polymers,” POLYMER SCIENCE AND ENGINEERING, Vol. 1, 2nd ed., pp. 234-268, John Wiley & Sons, (1984).

### 3. Poly(meth)acrylate Polymer (P(M)AP)

The active layer may include at least one polymethacrylate and/or polyacrylate polymer (P(M)AP). In the active layer of FIG. 1, the P(M)AP may be blended with the TPU and PSA and other components of layer 12. In the active layer of FIG. 3, a P(M)AP may be blended with the TPU and other components of the sub-layer 30 and/or with the PSA in sub-layer 32. The exemplary P(M)AP is different from the polymer(s) which constitute the PSA. The P(M)AP may be at least 2 wt. % of the polymer matrix, such as at least 5 weight %, or at least 10 weight %. The P(M)AP may be up to 50 wt. % of the polymer matrix, such as at up to 45 wt.

A ratio by weight of PSA:P(M)AP in the active layer 12, 12′ may be at least 3:1, such as at least 5:1, or at least 8:1 and may be up to 20:1, e.g., about 9:1. Including the P(M)AP in the polymer matrix improves certain physical properties of the matrix. The P(M)AP may act as a pH regulating agent to maintain the active pharmaceutical agent in an advantageous state. For example, a P(M)AP with acid functionality may maintain an acid active in the neutral acid form (e.g., stabilize diclofenac as an acid). A P(M)AP with amine functionality may maintain an amine active in the neutral base form (e.g., stabilize fentanyl as a base). An acid functional P(M)AP may be paired with an amine-based active to ensure the protonated active form is stabilized. A P(M)AP may also act as a hydrogen bonding source via neutral polar groups such as OH, and amide, e.g., vinylpyrrolidone, vinylpyrrole, vinylimidazole, vinylcaprolactam, vinyloxazole, to hydrogen bond to the TPU polymer. The P(M)AP may also have enhanced film forming capabilities to give better physical properties (cohesion) to the active layer.

A ratio by weight of the TPU:(PSA+P(M)AP) in the active layer 12, 12′ may be at least 1:4, such as from 1:3 to 3:1, or at least 1:1.2, or at least 1:1, or at least 2:1.

The P(M)AP may be derived from a blend of methacrylic and acrylic based monomers such that the properties of the polymer are not those of a PSA. P(M)AP having functional groups are homopolymers, copolymers or terpolymers which contain, monomer units having free functional groups further nonfunctional monomer units such as (meth)acrylate esters or (meth)acrylamide may also be present. The monomers can be monofunctional or polyfunctional. These functional groups include carboxyl groups, hydroxy groups, amino groups, amido groups, epoxy groups, etc.

Functional monomers that are polymerizable include acrylic acid, methacrylic acid, maleic acid, maleic anhydride, hydroxyethyl acrylate, hydroxypropyl acrylate, acrylamide, dimethylacrylamide, acrylonitrile, dimethyl aminoethyl acrylate, dimethyl aminoethyl methacrylate, tert-butyl aminoethyl acrylate, tert-butyl aminoethyl methacrylate, methoxyethyl acrylate and methoxyethyl methacrylate and other monomers having at least one unsaturated double bond which participates in copolymerization reaction in one molecule and a functional group on its side chain such as a carboxyl group, a hydroxyl group, a sulfoxyl group, an amino group, an pyrole group and an alkoxyl, etc. Further examples include monomers with at least one of the following moieties: alkylene, hydroxy-substituted alkylene, carboxylic acid-substituted alkylene, vinyl alkanoate, vinylpyrrolidone, vinylpyridine, vinylpyrazine, vinylpyrrole, vinylimidazole, vinylcaprolactam, vinyloxazole, vinyl acetate, vinyl propionate, and vinyl morpholine. Exemplary carboxyl functional monomers include acrylic acid, methacrylic acid, itaconic acid, maleic acid, and crotonic acid. Exemplary hydroxy functional monomers include 2-hydroxyethyl methacrylate, 2-hydroxyethyl acrylate, hydroxymethyl acrylate, hydroxymethyl methacrylate, hydroxyethyl acrylate, hydroxyethyl methacrylate, hydroxypropyl acrylate, hydroxypropyl methacrylate, hydroxybutyl acrylate, hydroxybutyl methacrylate, hydroxyamyl acrylate, hydroxyamyl methacrylate, hydroxyhexyl acrylate, hydroxyhexyl methacrylate. These functional monomers may be copolymerized with nonfunction (meth)acryl alkyl esters and (meth)acryl alkyl amides.

Example P(M)AP's include Eudragit® E100, Eudragit® L100 (Methacrylic Acid-Methyl Methacrylate Copolymer (1:1)), Eudragit® S100 (Methacrylic Acid-Methyl Methacrylate Copolymer (1:2)), Eudragit® RL100 ethyl acrylate-methyl methacrylate-methacrylic acid ester with quaternary ammonium groups), Eudragit® RS100, Eudragit® L100-55, Eudragit® E PO, Eudragit® RL PO, Eudragit® RS PO and the like, manufactured by Röhm Co. Ltd. (Germany), and Eudragit Nos. I, II, III and IV, manufactured by Jiangsu Lianyun'gang Iodine Factory.

In the Examples below, Eudragit® E100 is used as the P(M)AP. Eudragit® E100 is a cationic copolymer based on dimethylaminoethyl methacrylate, butyl methacrylate, and methyl methacrylate (specifically, poly(butyl methacrylate-co-(2-dimethylaminoethyl) methacrylate-co-methyl methacrylate) 1:2:1). However, similar acrylates/dimethylaminoethyl methacrylate copolymers can alternatively be used.

In one embodiment, the pressure-sensitive adhesive (e.g., acrylic pressure-sensitive polymer) is combined with one of the Eudragit® acrylic resins described above. Such an acrylic-based adhesive composition mixture is described in Example 4 of CN1640500A (Beijing Kangbeide Pharmaceuticals Co., Ltd), which consists of an acrylic pressure-sensitive polymer (e.g., PSA-10-K, an N,N-substituted methacrylate polymer) and Eudragit® E100.

A ratio, by weight, of the PSA (e.g., PSA-10-K):PMP (e.g., Eudragit® E100) may be from 1:1 to 20:1, such as at least 5:1 or at least 8:1, e.g., 9:1.

The PSA and/or P(M)AP may be incorporated into the components of, or into the reaction mixture for, the preparation of the TPU resin, or after making the TPU resin. In another process, all the materials can be mixed with the TPU resin and then melted or they can be incorporated directly into the melt of the TPU resin.

### 4. Other Polymeric Materials

Polyvinylpyrrolidone may be incorporated in the active layer and is available as povidone polymers, under the tradename Kollidon® from BASF in a wide range of molecular weights. The Kollidon® products are soluble and insoluble grades of polyvinylpyrrolidone (PVP) homo and copolymers of various molecular weights and particle sizes. The polymers are used as dry binders, film-formers, stabilizers in suspensions, dispersants, enzyme stabilizers, and for improvement of bioavailability. While not an adhesive in itself, polyvinylpyrrolidone can act as an adhesive activator. It may also be co polymerized with vinyl acetate.

In some embodiments, the polymer matrix (or entire active layer 12, 12′) is substantially free of (or even completely free of) polyisobutylene polymer. In some embodiments, the polymer matrix is substantially free of (or even completely free of) styrene-isoprene-styrene block copolymer.

### 5. The Active Agent

The exemplary device includes a composition for the delivery of an active agent, such as active pharmaceutical ingredient. The active agent may include an active pharmaceutical or cosmetic ingredient suitable for the treatment or care of the skin, or for treatment or care of a condition existing in the body below the skin. Example active agents include drugs or pharmaceutically acceptable salts thereof, and may be combined with one or more excipients.

The exemplary system is in the form of a flexible finite system for topical application, which includes a polymer matrix with a therapeutically effective amount of an active pharmaceutical ingredient therein.

The active pharmaceutical ingredient (e.g., a drug) may be present in an amount of from 1% to 50%, or from 1% to 40%, or from 4% to 40%, or from 4% to 30%, percent by weight of the active layer 12, 12′, or from 1%, 4%, 5%, or 10% up to 50%, 40%, 30%, 20%, 10%, or 5%, and further including at least 1%, or 2%, or 3%, or 4% or 5%, or 6%, or 7%, or 8%, or 9%, or about 10% by weight. These amounts are for the active ingredient alone (i.e., excluding any excipients with which the active agent may be mixed.

Active agents useful herein may be categorized or described herein by their therapeutic and/or cosmetic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated application or applications listed.

In some embodiments, the active pharmaceutical ingredient (API) includes at least one pharmaceutically active compound that has high permeation through the skin. In some embodiments, the active compound is of low molecular weight (below 500 daltons), has a Log P (Log partition coefficient) value between 1 and 4, and is a semi-hydrophobic molecule with a low melting point. Lipophilicity represents the affinity of a molecule or a moiety for a lipophilic environment. As used herein log P is measured by its distribution in a biphasic system (octanol-water).

The active agents may be selected from central nervous system (CNS) active agents, pain management agents, skin whitening or depigmenting agents, anti-acne agents, anti-wrinkle and/or anti-aging agents, agents stimulating healing, emollients, AQP-3 modulating agents, aquaporin modulating agents, proteins from the aquaporin family, collagen synthesis stimulating agents, agents modulating PGC-1-α synthesis, agents modulating the activity of PPARγ, agents which increase or reduce the triglyceride content of adipocytes, agents stimulating or delaying adipocyte differentiation, lipolytic agents or agents stimulating lipolysis, anti-cellulite agents, adipogenic agents, inhibitors of acetylcholine-receptor aggregation, agents inhibiting muscle contraction, anticholinergic agents, elastase inhibiting agents, matrix metalloproteinase inhibiting agents, melanin synthesis stimulating or inhibiting agents, propigmenting agents, self-tanning agents, NO-synthase inhibiting agents, 5α-reductase inhibiting agents, lysyl- and/or prolyl hydroxylase inhibiting agents, antioxidants (acting on the body rather than simply as preservatives of the composition), free radical scavengers and/or agents against atmospheric pollution, reactive carbonyl species scavengers, anti-glycation agents, antihistamine agents, antiviral agents, antiparasitic agents, skin conditioners, humectants, substances which retain moisture, alpha hydroxy acids, beta hydroxy acids, moisturizers, hydrolytic epidermal enzymes, vitamins, amino acids, proteins, biopolymers, agents able to reduce or treat the bags under the eyes, exfoliating agents, desquamating agents, keratolytic agents, antimicrobial agents, antifungal agents, fungistatic agents, bactericidal agents, bacteriostatic agents, agents stimulating the synthesis of dermal or epidermal macromolecules and/or capable of inhibiting or preventing their degradation, elastin synthesis-stimulation agents, decorin synthesis-stimulation agents, laminin synthesis-stimulation agents, defensin synthesis-stimulating agents, chaperone synthesis-stimulating agents, cAMP synthesis-stimulating agents, heat shock proteins, HSP70 synthesis stimulators, heat shock protein synthesis-stimulating agents, hyaluronic acid synthesis-stimulating agents, fibronectin synthesis-stimulating agents, sirtuin synthesis-stimulating agents, agents stimulating the synthesis of lipids and components of the stratum corneum, ceramides, fatty acids, agents that inhibit collagen degradation, agents that inhibit elastin degradation, agents that inhibit serine proteases, agents stimulating fibroblast proliferation, agents stimulating keratinocyte proliferation, agents stimulating adipocyte proliferation, agents stimulating melanocyte proliferation, agents stimulating keratinocyte differentiation, agents that inhibit acetylcholinesterase, skin relaxant agents, glycosaminoglycan synthesis-stimulating agents, antihyperkeratosis agents, comedolytic agents, anti-psoriasis agents, anti-dermatitis agents, anti-eczema agents, DNA repair agents, DNA protecting agents, stabilizers, anti-itching agents, agents for the treatment and/or care of sensitive skin, firming agents, redensifying agents, restructuring agents, anti-stretch mark agents, binding agents, agents regulating sebum production, coadjuvant healing agents, agents stimulating reepithelization, coadjuvant reepithelization agents, cytokine growth factors, calming agents, anti-inflammatory agents, anesthetic agents, agents acting on capillary circulation and/or microcirculation, agents stimulating angiogenesis, agents that inhibit vascular permeability, venotonic agents, agents acting on cell metabolism, agents to improve dermal-epidermal junction, agents inducing hair growth, hair growth inhibiting or retardant agents, chelating agents, plant extracts, essential oils, marine extracts, agents obtained from a biofermentation process, mineral salts, cell extracts, sunscreens and organic or mineral photoprotective agents active against ultraviolet A and/or B rays, and mixtures thereof.

Example pharmaceutically active agents that are skin permeable include:

1. Central nervous system (CNS) active agents, such as agents used to treat Alzheimer's, e.g., rivastigmine, donepezil, latrepirdine, and memantine; agents used to treat Parkinson's, such as rotigotine and lisuride; stimulants used to treat ADHD, such as methylphenidate, amphetamine base or salt, agents for treatment of depression, such as selegiline and agomelatine; agents used to treat CNS disorders, such as schizophrenia, e.g., olanzapine and blonanserin; agents used to treat migraine disorders, such as compounds of the triptan class, e.g., sumatriptan, zolmitriptan, and derivatives thereof.

2. Pharmaceutical pain management agents, including active agents for treating local pain including NSAIDs, such as naproxen, indomethacin, and copper-indomethacin combinations, ketoprofen, gabapentin, local anesthetics, such as lidocaine and salts such as lidocaine hydrochloride, prilocaine, tetracaine and combinations thereof, trolamine salicylate, and systemic pain relief agents, such as fentanyl, buprenorphine, and dronabinol, those providing counter irritant pain relief by cooling with menthol, menthol derivatives, camphor, those providing heating including capsaicin, and active agents for specific location pain such breast tenderness and nodularity by 4-hydroxytamoxifen, methylsalicylate and mixtures thereof, acetylsalicylic acid (aspirin) and other salicylic acid esters, diclofenac and salts thereof such as sodium, diethylamine and epolamine, ibuprofen, ketoprofen, acetaminophen and other non-steroidal anti-inflammatory drugs, and the like, and combinations thereof.

3. Antiviral medications, such as acyclovir (cold sore, herpes simplex).

4. Antiemetics for treating nausea, such as dronabinol, ondansetron, prochlorperazine, phenothiazines, scopolamine, and ganisetron.

5. Hypertension API's, such as bisoprolol, clonidine and enalapril and derivatives such as enalapril maleate and enalaprilat ethyl ester.

6. Hormones, such as estradiol, ethynyl estradiol, norelgestromin, levonorgestrel, levonorgestrel acetate, testosterone, gonadotropin-releasing hormone.

7. Smoking cessation therapies, such as nicotine.

8. Skin-whitening or depigmentation agents, including ascorbic acid, hydrogen peroxide, magnesium ascorbyl phosphate, aminopropyl ascorbyl phosphate, pyridine-3-carboxamide (nicotinamide), kojic acid, hydroquinine, mulberry root extract, liquorice root extract, Scutellaria baicalensis extract, grape extract, ferulic acid, hinokitiol, arbutin, α-arbutin (bearberry extract), and mixtures thereof.

9. Anti-acne agents, such as salicylic acid, glycolic acid, lactobionic acid, azelaic acid, benzoyl peroxide, antibiotics such as clindamycin, sodium sulfacetamide and erythromycin, retinoids such as adapalene, tazarotene, and tretinoin, which may be sold under trade names such as Retin-A®, Differin®, Renova®, and Tazorac®, and mixtures thereof.

10. Anti-wrinkle agents and/or anti-aging agents, such as extracts or hydrolyzed extracts of Vitis vinifera, Rosa canina, Curcuma longa, Theobroma cacao, Ginkgo biloba, Leontopodium alpinum or Dunaliella salina, hydrolyzed Hibiscus esculentus extract (e.g., Myoxinol™), Cassia alata leaf extract (e.g., DN AGE™ LS, marketed by Laboratoires Serobiologiques/Cognis), locust bean (Ceratonia siliqua) gum (e.g., Phytaluronate), Oryza sativa (Rice) Extract (e.g., Orsirtine™ GL [INCI name: Water, Glycerin, and Oryza Sativa (Rice) Extract], from ISP Vincience), Phoenix dactylifera (Date) Seed Extract (e.g., D'Orientine™ IS, from Vincience/ISP), Einkorn (Triticum monococcum) extract (e.g., Phytoquintescine™, from ISP Vincience), Acmella oleracea extract (e.g., Gatuline® Expression, from Gattefossé), Spilanthes acmella flower extract (e.g., Gatuline® In-Tense, from Gattefossé), Juglens regia (Walnut) Seed Extract (e.g., Gatuline® Age Defense 2, from Gattefossé, algae extract (e.g., Thalassine™, from Biotechmarine), Pyrus malus fruit extract (e.g., EquiStat™ [INCI: Pyrus malus Fruit Extract, Glycine soja Seed Extract], from Coletica/Engelhard), Malus domestica fruit cell culture (e.g., PhytoCellTec Malus Domestica, marketed by Mibelle Biochemistry, Pimpinella anisum extract (e.g., Bioxilift®, from Nivea), Annona squamosa seed extract (e.g., SMS Anti-Wrinkle® from Silab); synthetic compounds or products, such as polypeptides (using INCI names), e.g., di-, tri-, tetra, penta, hexa-, hepta, and octo-polypeptides, such as dipeptide-4 (e.g., Quintescine™ IS from ISP Vincience), tripeptide-1 (e.g., Kollaren™ [INCI: Tripeptide 1, Dextran], from Lucas Meyer Cosmetics, or Aldenine® [INCI: Tripeptide 1, hydrolyzed wheat protein, hydrolyzed soy protein palmitoyl]), pentapeptide-3 (Gly-Pro-Arg-Pro-Ala, e.g., Vialox™), pentapeptide-18 (e.g., Leuphasyl®, from Lipotec), hexapeptide-9 (e.g., Collaxyl™ IS, from Ashland), heptapeptide (e.g., Laminixyl 15™ from Ashland); palmitoyl, caproyl, and acetyl polypeptides containing from 2-10 amino acid residues, such as palmitoyl oligopeptide (e.g., Matrixyl 3000™ a mixture of palmitoyl oligopeptide and palmitoyl tetrapepide-7 (Palm-Val-Gly-Val-Ala-Pro-Gly), or Dermaxyl™, both from Sederma), palmitoyl tripeptide-5 (e.g., Syn™-Coll, by Pentapharm/DSM), palmitoyl tetrapeptide-7 (e.g., in Matrixyl 3000™) palmitoyl pentapeptide-4 (Palm-Lys-Thr-Thr-Lys-Ser, e.g., Matrixyl™, from Sederma), palmitoyl hexapeptide-19 (e.g., BONT-L-Peptide, from Infinitec Activos), caprooyl tetrapeptide-3 (e.g., ChroNOline™, from Lucas Meyer Cosmetics), acetyl tetrapeptide-2 (e.g., Thymulen™-4 from Lucas Meyer Cosmetics), acetyl tetrapeptide-11 (e.g., Syniorage™, from BASF), acetyl tetrapeptide-22 (e.g., Thermostressine®, from Lipotec), acetyl tetrapeptide-9 (e.g., Dermican™, from BASF), acetyl tetrapeptide-5 (e.g., Eyeseryl®, from Lipotec), acetyl hexapeptide-8 (e.g., Argireline®, from Lipotec), acetyl hexapeptide-30 (e.g., Inyline®, from Lipotec), acetyl octapeptide-3 (SNAP-8, from Lipotec), diaminopropionoyl tripeptide-33 (e.g., Preventhelia®, from Lipotec), tripeptide-9 citrulline (e.g., dGlyage® [INCI: Lysine HCl, Lecithin, Tripeptide-9 Citrulline], from Lipotec), tripeptide-10 citrulline (e.g., Decorinyl®, from Lipotec), acetyl tripeptide-30 citrulline (Peptide AC29), tripeptide-10 citrulline (e.g., Vilastene® [INCI: Lysine HCl, Lecithin, Tripeptide-10 Citrulline], from Lipotec), acetylarginyltriptophyl diphenylglycine (e.g., Relistase®, from Lipotec), methylsilanol mannuronate (e.g., Algisum C™, from Exsymol), methylsilanol hydroxyproline aspartate (e.g., Hydroxyprolisilane CN™, from Exsymol), dimethylmethoxy chromanol (e.g., Lipochroman®-6, from Lipotec), dimethylmethoxy chromanyl palmitate (e.g., Chromabright®, from Lipotec), Pseudoalteromonas Ferment Extract (e.g., Trylagen® [INCI: Pseudoalteromonas ferment extract, hydrolyzed wheat protein, hydrolyzed soy protein, tripeptide 10 citrulline, tripeptide 1], Antarcticine®, and Hyadisine®, all from Lipotec), palmitoyl hydrolyzed wheat protein (e.g., Deepaline™ PVB, from Seppic), dipalmitoyl hydroxyproline (e.g., Sepilift™ DPHP, from Seppic), mixtures such as Juvenesce [INCI: Ethoxydiglycol and Caprylic Triglyceride, Retinol, Ursolic Acid, Phytonadione, Ilomastat], marketed by Coletica/Engelhard/BASF), Ameliox™ [INCI: Carnosine, Tocopherol, Silybum marianum Fruit Extract], and mixtures thereof.

11. Antagonists of the Ca2+ channel such as alverine, manganese or magnesium salts, certain secondary or tertiary amines, retinol and its derivatives, idebenone and its derivatives, Coenzyme Q10 and its derivatives, boswellic acid and its derivatives, GHK and its derivatives and/or salts, carnosine and its derivatives, DNA repair enzymes such as photolyase or T4 endonuclease V, or chloride channel agonists, and mixtures thereof. calcium hydroxymethionine (e.g., Essenskin™) teprenone (e.g., Renovage™), dipeptide diaminobutyroyl benzylamide diacetate (e.g., Syn™ Ake™, marketed by Sederma/Croda), Glycine soja (Soybean) Protein with Oxido Reductases (e.g., Preregen™, marketed by Pentapharm/DSM),

12. Moisturizing agents, such as glycerin, hyaluronic acid, urea, trehalose, sodium hyaluronate, sodium chondroitin sulfate, sodium lactate, sodium pyrrolidone carboxylate, betaine, lactic acid bacteria fermented solution, yeast extract, and mixtures thereof.

13. Anti-inflammatory agents, such as spike moss extract, seal whip extract, Polygonum cuspidatum root extract, and mixtures thereof.

14. DNA repair agents, such as C₁-C₈ alkyl tetrahydroxycyclohexanoate, micrococcus lysate, bifida ferment lysate, and mixtures thereof.

14. Skin lipid barrier repair agents, such as phytosphingosine, linoleic acid, cholesterol, and mixtures thereof.

15. Anti-cellulite agents, such as Coleus forskohlii root extract, Magnolia grandiflora bark extract, Nelumbo nucifera leaf extract, and mixtures thereof.

16. Wound-healing agents, such as Mimosa tenuiflora bark extract, soybean protein, and mixtures thereof.

17. Hair growth retardation agents, such as ursolic acid, phytosphingosine, Boswellia serrata extract, and mixtures thereof.

18. Hair growth stimulating agents, such as Serenoa serrulata fruit extract, licorice extract, acetyl glucosamine, and mixtures thereof.

19. Agents for reducing bags under the eye and dark circles, such as hesperidin methyl chalcone, dipeptide-2, Passiflora incarnate flower extract, linoleic acid, isolinoleic acid, and mixtures thereof.

20. Collagen synthesis or blood circulation enhancing agents, such as arginine, Ascophyllum nodosum extract, Asparagopsis armata extract, caffeine, and mixtures thereof.

21. Antioxidants, such as nordihydroguaiaretic acid, grape seed extract, green tea leaf extract, butylhydroxyanisole, butylhydroxytoluene, propyl gallate, erythorbic acid, sodium erythorbate, para-hydroxyanisole, octyl gallate, phenolic type antioxidants, organic phosphites, phosphines and phosphonites, hindered amines, organic amines, organo sulfur compounds, lactones, hydroxylamine compounds, and mixtures thereof.

22. Ultraviolet ray absorbers, such as: p-aminobenzoic acid and derivatives thereof, cinnamic acid derivatives such as benzyl cinnamate, benzophenone derivatives such as 2,4-dihydroxybenzophenone, salicylic acid derivatives such as benzyl salicylate, and mixtures thereof.

22. Amino acids and naturally occurring peptides which do not, of themselves, have a therapeutic effect, such as glycine, alanine, valine, leucine, isoleucine, serine, threonine, phenylalanine, tyrosine, tryptophan, cystine, cysteine, methionine, proline, hydroxyproline, aspartic acid, asparagine, glutamic acid, glutamine, arginine, histidine, lysine, γ-aminobutyric acid, wheat peptides and soybean peptide, and mixtures thereof.

23. Vitamins and factors acting like a vitamin, such as vitamin A and analogues thereof such as retinol and retinoic acid, carotenoids such as α-carotene and β-carotene, vitamin B₁ and analogues thereof such as thiamines, vitamin B2 and analogues thereof such as riboflavin, vitamin B6 and analogues thereof such as pyridoxine, vitamin B12 and analogues thereof such as cyanocobalamin, folic acid, nicotinic acid, pantothenic acid, vitamin C and analogues thereof such as L-ascorbic acid, vitamin D and analogues thereof such as ergocalciferol and cholecalciferol, vitamin E and analogues thereof such as d-α-tocopherol and γ-tocopherol, Coenzyme Q10, vitamin K and analogues thereof, carnitine, ferulic acid, α-lipoic acid, orotic acid, and mixtures thereof.

24. Antiseptic agents, such as benzoic acid, sodium benzoate, undecylenic acid, salicylic acid, sorbic acid, potassium sorbate, dehydroacetic acid, sodium dehydroacetate, isobutyl p-oxybenzoate, isopropyl p-oxybenzoate, ethyl p-oxybenzoate, butyl p-oxybenzoate, propyl p-oxybenzoate, benzyl p-oxybenzoate, methyl p-oxybenzoate, sodium p-oxybenzoate methyl, phenoxyethanol, light sensitive dye No. 101, light sensitive dye No. 201 and light sensitive dye No. 401, and mixtures thereof.

25. Chelating agents, such as ethylene diamine tetraacetic acid, trisodium ethylenediamine hydroxyethyl triacetate, sodium citrate, gluconic acid, phytic acid, sodium polyphosphate, sodium metaphosphate, and mixtures thereof.

26. Anti-inflammatory agents, such as glycyrrhizinic acid, and sodium, potassium and ammonium salts thereof, lysozyme chloride, hydrocortisone, allantoin, and mixtures thereof.

These active agents may in the form of pharmaceutically acceptable salts, prodrugs, and/or derivatives thereof, and any combination thereof of the above.

In one embodiment, the active agent includes a tertiary amine compound, such as a drug, which may be selected from the group consisting of amiodarone, amitriptyline, atropine, benztropine, biperiden, bornaprine, bupivacaine, chlorpheniramine, cinnarizine, clomipramine, cyclopentolate, darifenacin, dexetimide, dicyclomine, diltiazem, diphenhydramine, doxepin, ethopropazine, fentanyl, flavoxate, homatropine, imipramine, loxapine, mazaticol, metixene, oxybutin, oxyphencyclimine, phenglutarimide, physostigmine, piperidolate, pirenzepine, procyclidine, profenamine, propiverine, rivastigmine, rotigotine, scopolamine, telenzepine, theophylline, tolterodine, trimipramine, trihexyphenidyl, tropatepine, tropicamide, rivastigmine, and mixtures thereof, donepezil, memantine, fentanyl, oxybutynin, rotigotine, ropinirole, rivastigmine, tamsulosin, methylphenidate, fingolimod, and buprenorphine.

In another embodiment, the active agent includes at least one of agomelatine, amphetamine, scopolamine, levonorgestrel, ethinyl estradiol, levonorgestrel acetate, testosterone, methylphenidate, enalaprilat ethyl ester, clonidine, ketoprofen, nicotine, lidocaine, lidocaine HCL, pharmaceutically acceptable salts, prodrugs, and/or derivatives thereof, and any combination thereof.

### 7. Excipients

Excipients may be incorporated in the active layer for long-term stabilization, as fillers or diluents, to confer a therapeutic enhancement on the active ingredient in the final dosage form, such as facilitating drug absorption, for enhancing adhesion of the PSA, for enhancing solubility of the active agent, to aid in the handling of the active agent during processing.

Example excipients which may be present include lubricants, organic solvents, binders, thickeners, emulsifiers, surfactants, fillers, pigments, dyes, perfumes, preservatives, penetration enhancers, and combinations thereof. While the poly(meth)acrylate polymer may act as an excipient, it is not considered as an excipient for purposes of calculating weights of excipients present.

Components serving as lubricants, solvents, binders and thickeners and emulsifiers may include one or more of liquid hydrocarbons, waxes, natural fats and fatty oils, alcohols, ethers, esters, silicone oils, monosaccharides, polymers, and the like.

Exemplary solvents include class Ill solvents, such as ethylacetate, heptane isopropyl acetate, methylethylketone, ethanol, butanol, butyl acetate, and DMSO and oligomeric α-olefins, C₆-C₄₀ alkanes and cycloalkane, C₁₀-C₄₀ alkenes, and mixtures thereof, such as squalene, ceresin, mineral oils, and petroleum jelly.

Exemplary waxes include microcrystalline wax, natural waxes such as jojoba oil, carnauba wax, candelilla wax, rice bran wax, shellac, lanolin, mink sebaceous wax, spermaceti wax, sugarcane wax, sperm whale oil, beeswax and montan wax.

Exemplary natural fats and fatty oils include avocado oil, almond oil, olive oil, extra virgin olive oil, sesame seed oil, rice bran oil, rice oil, rice germ oil, corn oil, safflower oil, soybean oil, maize oil, rape seed oil, persic oil, palm kernel oil, palm oil, castor oil, sunflower oil, high oleic sunflower oil, grape seed oil, cottonseed oil, coconut oil, hydrogenated coconut oil, beef tallow, hydrogenated oil, horse oil, mink oil, yolk oil, yolk fat oil, rose hip oil, kukui nut oil, evening primrose oil, wheat germ oil, peanut oil, Camellia japonica oil, Camellia kissi oil, cacao butter, Japan wax, beef bone tallow, neat's-foot oil, swine tallow, equine tallow, ovine tallow, shea butter, macadamia nut oil and meadow foam seed oil.

Exemplary fatty acids include lauric acid, myristic acid, palmitic acid, stearic acid, behenic acid, oleic acid, linoleic acid, linolenic acid, γ-linolenic acid, isostearic acid, 12-hydroxystearic acid, undecenoic acid and coconut oil fatty acid.

Exemplary lower alcohols include ethanol, 1-propanol, 2-propanol, 1-butanol, 2-butanol and benzyl alcohol. Exemplary higher alcohols include isostearyl alcohol, 2-octyldodecan-1-ol, 2-hexyldecan-1-ol, cholesterol, phytosterol, lauryl alcohol, myristyl alcohol, cetyl alcohol, stearyl alcohol, oleyl alcohol, behenyl alcohol and cetostearyl alcohol. Exemplary polyhydric alcohols include ethylene glycol, diethylene glycol, triethylene glycol, polyethylene glycol, propylene glycol, dipropylene glycol, polypropylene glycol, pentanediol, glycerin, diglycerin, polyglycerin, isoprene glycol, 1,3-butylene glycol, 3-methyl-1,3-butanediol, 1,3-butanediol, 1,2-pentanediol and 1,2-hexanediol.

Exemplary alkylglyceryl ethers include stearyl monoglyceride, 3-hexadecoxypropane-1,2-diol, 3-[(Z)-octadec-9-enoxy]propane-1,2-diol and isostearyl glyceryl ether.

Exemplary esters include isopropyl myristate, butyl myristate, isopropyl palmitate, ethyl stearate, butyl stearate, ethyl oleate, ethyl linoleate, isopropyl linoleate, cetyl caprylate, hexyl laurate, isooctyl myristate, decyl myristate, myristyl myristate, cetyl myristate, octadecyl myristate, cetyl palmitate, stearyl stearate, decyl oleate, oleyl oleate, cetyl ricinoleate, isostearyl laurate, isotridecyl myristate, isocetyl myristate, isostearyl myristate, 2-octyldodecyl myristate, 2-ethylhexyl palmitate, isocetyl palmitate, isostearyl palmitate, 2-ethylhexyl stearate, isocetyl stearate, isodecyl oleate, octyldodecyl oleate, octyldodecyl ricinoleate, ethyl isostearate, isopropyl isostearate, cetyl 2-ethylhexanoate, cetostearyl 2-ethylhexanoate, stearyl 2-ethylhexanoate, hexyl isostearate, ethylene glycol dioctanoate, ethylene glycol dioleate, propylene glycol dicaprylate, propylene glycol dicaprylate/dicaprate, lauryl lactate, myristyl lactate, cetyl lactate, trioctyl citrate, diisostearyl malate, 2-ethylhexyl hydroxystearate, diisopropyl adipate, diisopropyl sebacate, dioctyl sebacate, cholesteryl stearate, cholesteryl isostearate, cholesteryl hydroxystearate, cholesteryl oleate, dihydrocholesteryl oleate, phytosteryl isostearate, phytosteryl oleate, isocetyl 12-stearoyl hydroxystearate, stearyl 12-stearoyl hydroxystearate, isostearyl 12-stearoyl hydroxystearate, octyl isononanoate.

Exemplary silicone oils include polysiloxanes, polyether modified silicones, alcohol modified silicones, alkyl modified silicones, and amino modified silicones.

Exemplary saccharides include mannitol, sorbitol, xylitol, maltitol, erythritol, pentaerythritol, glucose, sucrose, fructose, lactose, maltose, xylose and trehalose.

Exemplary polymers include sodium alginate, carrageenan, agar, guar gums, tamarind gum, dextrin, starch, locust bean gum, gum arabic, pectin, quince, chitosan, starch, curdlan, xanthan gum, dextran, pullulan, microcrystalline cellulose, methyl cellulose, ethyl cellulose, hydroxyethyl cellulose, hydroxypropyl cellulose, hydroxypropyl methylcellulose, carboxymethyl cellulose, carboxy starch, cationized cellulose, starch phosphate ester, albumin, casein, and gelatin.

Exemplary anionic surfactants include potassium coconut oil fatty acid, sodium coconut oil fatty acid, triethanolamine coconut oil fatty acid, potassium laurate, sodium laurate, triethanolamine laurate, potassium myristate, sodium myristate, isopropanolamine myristate, potassium palmitate, sodium palmitate, isopropanolamine palmitate, potassium stearate, sodium stearate, triethanolamine stearate, potassium oleate, sodium oleate, castor oil fatty acid sodium, zinc undecylate, zinc laurate, zinc myristate, magnesium myristate, zinc palmitate, zinc stearate, calcium stearate, magnesium stearate, aluminum stearate, calcium myristate, magnesium myristate, aluminum dimyristate, aluminum isostearate, polyoxyethylene lauryl ether acetate, sodium polyoxyethylene lauryl ether acetate, polyoxyethylene tridecyl ether acetate, sodium polyoxyethylene tridecyl ether acetate, sodium stearoyl lactate, sodium isostearoyl lactate, sodium lauroyl sarcosinate, coconut oil fatty acid sarcosinate, sodium coconut oil fatty acid sarcosinate, coconut oil fatty acid sarcosine triethanolamine, lauroyl sarcosine, potassium lauroyl sarcosinate, lauroyl sarcosine triethanolamine, oleoyl sarcosine, sodium myristoyl sarcosinate, sodium stearoyl glutamate, coconut oil fatty acid acyl glutamic acid, potassium coconut oil fatty acid acyl glutamate, sodium coconut oil fatty acid acyl glutamate, lauroyl glutamic acid, potassium lauroyl glutamate, sodium lauroyl glutamate, myristoyl glutamic acid, potassium myristoyl glutamate, sodium myristoyl glutamate, stearoyl glutamic acid, potassium stearoyl glutamate, disodium stearoyl glutamate, sodium hydrogenated beef tallow fatty acid acyl glutamate, sodium coconut oil fatty acid/hydrogenated beef tallow fatty acid acyl glutamate, lauroyl methyl alanine, sodium lauroyl methyl alanine, sodium myristoyl methyl alanine, sodium lauroyl methyl taurate, sodium oleoyl methyl taurate, sodium alkane sulfonate, sodium tetradecene sulfonate, sodium dioctyl sulfosuccinate, disodium lauryl sulfosuccinate, sodium coconut oil fatty acid ethyl ester sulfonate, sodium lauryl sulfate, triethanolamine lauryl sulfate, sodium cetyl sulfate, triethanolamine alkyl sulfates, sodium alkyl sulfates, triethanolamine alkyl sulfates, alkyl ammonium sulfates, diethanolamine alkyl sulfates, triethanolamine alkyl sulfates, triethanolamine alkyl sulfates, lauryl ammonium sulfate, potassium lauryl sulfate, magnesium lauryl sulfate, monoethanolamine lauryl sulfate, diethanolamine lauryl sulfate, sodium myristyl sulfate, sodium stearyl sulfate, sodium oleyl sulfate, triethanolamine oleyl sulfate, sodium polyoxyethylene lauryl ether sulfates, triethanolamine polyoxyethylene lauryl ether sulfate, sodium polyoxyethylene alkyl ether sulfates, triethanolamine polyoxyethylene alkyl ether sulfates, sodium polyoxyethylene myristyl ether sulfates, sodium higher fatty acid alkanolamide sulfate esters, lauryl phosphate, sodium lauryl phosphate, potassium cetyl phosphate, diethanolamine cetyl phosphate, polyoxyethylene oleyl ether phosphate, polyoxyethylene lauryl ether phosphate, sodium polyoxyethylene lauryl ether phosphate, polyoxyethylene cetyl ether phosphate, sodium polyoxyethylene cetyl ether phosphate, polyoxyethylene stearyl ether phosphate, polyoxyethylene oleyl ether phosphate, sodium polyoxyethylene oleyl ether phosphate, polyoxyethylene alkylphenyl ether phosphates, sodium polyoxyethylene alkylphenyl ether phosphates, triethanolamine polyoxyethylene alkylphenyl ether phosphates, polyoxyethylene octyl ether phosphate, polyoxyethylene alkyl ether phosphate, triethanolamine polyoxyethylene lauryl ether phosphate, and diethanolamine polyoxyethylene oleyl ether phosphate.

Exemplary cationic surfactants include alkyl amines, alkyl imidazolines, ethoxylated amides, quaternary compounds, quaternized esters, and alkyl amine oxides. Examples include lauramine oxide, dicetyldimonium chloride, and cetrimonium chloride.

Exemplary amphoteric and zwitterionic surfactants include betaines, alkyl amidopropyl betaines, alkyl sulfobetaines, alkyl glycinates, alkyl carboxyglycinates, alkyl amphopropionates, alkyl amidopropyl hydroxysultaines, acyl taurates and acyl glutamates wherein the alkyl and acyl groups have from 8 to 18 carbon atoms. Examples include cocoamidopropyl betaine, sodium cocoamphoacetate, cocoamidopropyl hydroxysultaine, and sodium cocoamphopropionate.

Exemplary nonionic surfactants include aliphatic (C₆-C₁₈) primary or secondary linear or branched chain acids, alcohols or phenols, alkyl ethoxylates, alkyl phenol alkoxylates (especially ethoxylates and mixed ethoxy/propoxy), block alkylene oxide condensate of alkyl phenols, alkylene oxide condensates of alkanols, ethylene oxide/propylene oxide block copolymers, semi-polar nonionics (e.g., amine oxides), as well as alkyl amine oxides. Other suitable nonionics include mono or di alkyl alkanolamides and alkyl polysaccharides, sorbitan fatty acid esters, polyoxyethylene sorbitan fatty acid esters, polyoxyethylene sorbitol esters, polyoxyethylene acids, and polyoxyethylene alcohols. Examples of nonionic surfactants include alkyl polyglucoside, cocamidopropyl and lauramine oxide, polysorbate 20, ethoxylated linear alcohols, cetearyl alcohol, lanolin alcohol, stearic acid, glyceryl stearate, polyoxyethylene lauryl ether, polyoxyethylene oleyl ether, PEG-100 stearate, sorbitan monooleate, sorbitan isostearate, and oleth 20, and mixtures thereof.

Exemplary powdered fillers and reinforcing agents include clay (kaolin), silicic anhydride, titanium dioxide, magnesium aluminum silicate, sericite, talc, boron nitride, mica, montmorillonite, alumina cellulose powder, carbon black, wheat starch, silk powder, maize starch, and mixtures thereof.

Exemplary dyes and pigments include nitro dyes, azo dyes, nitroso dyes, xanthene dyes, quinoline dyes, anthraquinone dyes, indigo dyes, sepia powder, caramel, cochineal, carbon black, yellow iron oxide, black iron oxide, red iron oxide, titanium oxide, titanium dioxide, and mixtures thereof.

Exemplary α-hydroxy acids include citric acid, glycolic acid, tartaric acid and lactic acid, and mixtures thereof.

Exemplary essential oils include Archangelica officinalis (angelica) oil, Canangium odoratum (ylang-ylang) oil, Canarium luzonicum (elemi) oil, orange oil, Chamomilla recutita (matricaria) oil, Anthemis nobilis oil, Elettaria cardamom (cardamom) oil, Acorus calamus (calamus) oil, Ferula galbaniflua (galbanum) oil, Cinnamomum camphora (camphor) oil, Daucus carota (carrot) seed oil, Salvia sclarea (clary sage) oil, Citrus paradisi (grapefruit) oil, Eugenia caryophyllus (clove) oil, Cinnamon bark oil, Coriandrum sativum (coriander) oil, Cupressus sempervirens (cypress) oil, Santalum album (sandalwood) oil, Juniperus virginiana (cedarwood) oil, Cymbopogon nardus (citronella) oil, Cinnamomum zeylanicum (Cinnamon) leaf oil, Jasmine officinale (jasmine) absolute oil, Juniperus communis (juniper Berry) oil, Zingiber officinale (ginger) extract, Mentha spicata (spearmint) oil, Salvia officinalis (sage) oil, cedar oil, Pelargonium graveolens (geranium) oil, Thymus vulgaris (thyme) oil, Melaleuca alternifolia (tea tree) oil, Myristica fragrans (nutmeg) oil, Melaleuca viridiflora (niaouli) oil, Citrus aurantium (neroli) oil, pine oil, Ocimum basilicum (basil) oil, Mentha arvensis oil, Pogostemon patchouli (patchouli) oil, Cymbopogon martini (palmarosa) oil, Foeniculum vulgare (fennel) oil, Citrus bigaradia (petitgrain) oil, Piper nigrum (black pepper) oil, Boswellia carterii (frankincense) oil, Vetiveria zizanioides (vetiver) oil, Mentha piperita (peppermint) oil, Citrus bergamia (bergamot) oil, benzoin oil, Aniba rosaeodora (rosewood) oil, Origanum majorana (marjoram) oil, mandarin oil, Commiphora myrrha (myrrh) oil, Melissa officinalis (balm mint) oil, Eucalyptus globulus oil, Citrus junos oil, Citrus aurantifolia (lime) oil, Ravensara aromatica (ravensara) oil, Lavandula latifolia (lavandin) oil, Lavandula angustifolia (lavender) oil, Tilia vulgaris (linden) oil, lemon oil, lemon grass oil, rose oil, Aniba rosaeodora (rosewood) oil, Rosmarinus officinalis (rosemary) oil and Levisticum officinale (lovage) oil, and mixtures thereof.

The compositions described herein may further comprise one or more penetration enhancers. A “penetration enhancer” is an agent known to accelerate the delivery of the drug through the skin. These agents also have been referred to as accelerants, adjuvants, and sorption promoters, and are collectively referred to herein as “penetration enhancers.” This class of agents includes those with diverse mechanisms of action, including those which have the function of improving percutaneous absorption, for example, by changing the ability of the stratum corneum to retain moisture, softening the skin, improving the skin's permeability, acting as penetration assistants or hair-follicle openers or changing the state of the skin including the boundary layer. In specific embodiments, the enhancer(s) serve to both enhance penetration of the drug through the stratum corneum and retain the drug at a site local to administration.

Illustrative penetration enhancers include sulfoxides, alcohols, fatty acids, fatty acid esters, polyols, amides, surfactants, terpenes, alkanones, and organic acids, among others.

Examples include polyhydric alcohols such as dipropylene glycol, propylene glycol, and polyethylene glycol; oils such as olive oil, squalene, and lanolin; fatty ethers such as cetyl ether and oleyl ether; fatty acid esters such as isopropyl myristate; urea and urea derivatives such as allantoin which affect the ability of keratin to retain moisture; polar solvents such as dimethyl decyl phosphoxide, methyl octyl sulfoxide, dimethyl lauryl amide, dodecyl pyrrolidone, isosorbitol, dimethyl acetonide, dimethyl sulfoxide, decyl methyl sulfoxide, and dimethyl formamide which affect keratin permeability; salicylic acid which softens the keratin; amino acids which are penetration assistants; benzyl nicotinate which is a hair follicle opener; and higher molecular weight aliphatic surfactants such as lauryl sulfate salts which change the surface state of the skin and drugs administered. Other agents include oleic and linoleic acids, ascorbic acid, panthenol, butylated hydroxytoluene, tocopherol, tocopheryl acetate, tocopheryl linoleate, propyl oleate, and isopropyl palmitate. In some embodiments, combinations of two or more enhancers are used.

In some embodiments the penetration enhancers include one or more of:
(i) acyclic alcohols, including diols, triols, and glycols, such as ethanol, 1-propanol, 2-propanol, 1-butanol, 2-butanol, 1-pentanol, 2-pentanol, hexanol, octanol, nonanol, decanol, ethylene glycol, triethylene glycol, diethylene glycol, propylene glycol, dipropylene glycol, ethoxydiglycol, glycerol, propanediol, butanediol, pentanediol, 1,2,6-hexanetriol, stearyl alcohol, oleyl alcohol, linoleyl alcohol, linolenyl alcohol, caprylic alcohol, lauryl alcohol, myristyl alcohol, cetyl alcohol and polyethylene glycol;
(ii) ether alcohols, such as cyclic alcohols, menthol, 2-(2-hydroxypropan-2-yl)-5-methylcyclohexan-1-ol (Coolact®38 D), menthoxypropandiol (Coolact®10, available from Vantage Specialty Ingredients); ethers, and diethylene glycol monoethyl ether (e.g., sold under the tradenames Coolact®P, Transcutol®P, Transcutol®HP, and Transcutol®CG);
(iii) aromatic alcohols, such as benzyl alcohol;
(iv) fatty acids, such as capric acid, lauric acid, myristic acid, stearic acid, oleic acid, caprylic acid, valeric acid, heptanoic acid, pelargonic acid, hexanoic acid, isovaleric acid, neopentanoic acid, trimethyl hexanoic acid, neodecanoic acid, isostearic acid, neoheptanoic acid, and neononanoic acid;
(v) esters, including esters of fatty acids, such as isopropyl n-decanoate, isopropyl palmitate, octyldodecyl myristate, ethyl acetate, butyl acetate, methyl acetate, isopropyl n-butyrate, ethyl valerate, methyl propionate, diethyl sebacate, ethyl oleate, isopropyl n-hexanoate, isopropyl myristate, and fatty acid esters of (2-hydroxyethyl)-2-pyrrolidone;
(vi) aliphatic and cyclic amides, imidazoles, and sulfoxides, such as urea; mono and dialkyl amides, such as dimethylacetamide, diethyltoluamide, dimethylformamide, dimethyl octamide, dimethyl decamide, hexamethylene lauramide; 2-pyrrolidone and its derivatives, such as 1-hexyl-4-methoxycarbonyl-2-pyrrolidone, 1-lauryl-4-carboxy-2-pyrrolidone, 1-methyl-4-carboxy-2-pyrrolidone, 1-alkyl-4-imidazoline-2-one, 1-methyl-2-pyrrolidone, 2-pyrrolidone, 1-lauryl-2-pyrrolidone, 1-hexyl-4-carboxy-2-pyrrolidone, 1-methyl-4-methoxycarbonyl-2-pyrrolidone, 1-lauryl-4-methoxycarbonyl-2-pyrrolidone, N-cocoalkyl pyrrolidone, N-tallowalkyl pyrrolidone, N-dimethylaminopropyl pyrrolidone, N-cyclohexyl pyrrolidone; 2-caprolactam and its derivatives, such as 1-farnesylazacycloheptan-2-one, 1-farnesylazacyclopentan-2-one, 1-geranylazacycloheptan-2-one, 1-geranylgeranylazacycloheptan-2-one, 1-geranylazacyclohexane-2-one, 1-dodecylazacycloheptane-2-one (Azone®), 1-(3,7-dimethyloctyl)azacycloheptan-2-one, 1-(3,7,11-trimethyldodecyl)azacycloheptan-2-one, and 1-geranylazacyclopentan-2,5-dione; dimethyl sulfoxide, decyl methyl sulfoxide, dimethyl-acetamide, and dimethyl formamide;
(vii) alkanolamines, such as diethanolamine, triethanolamine, and derivatives thereof;
(viii) cationic surfactants, such as benzalkonium chloride, sodium laurate, sodium lauryl sulfate, cetylpyridinium chloride, citric acid, succinic acid, salicylic acid, salicylate, cetyl trimethyl ammonium bromide, tetradecyltrimethylammonium bromide, octadecyltrimethylammonium chloride, dodecyltrimethylammonium chloride, and hexadecyltrimethylammonium chloride;
(ix) nonionic surfactants, such as poloxamers (polyoxyethylene-polyoxypropylene-polyoxyethylene block copolymers), such as Poloxamer 231, Poloxamer 182, Poloxamer 184; polyethylene glycol alkyl ethers, such as Brij® 30, Brij® 35, Brij® 93, Brij® 96, Span® 99, Myrj®45, Myrj®51, Myrj®52; propylene glycol diesters, such as Miglyol®840; polyoxyethylene sorbitan esters and their derivatives, such as Tween® 20, Tween® 21, Tween® 40, Tween® 60, Tween® 61, Tween 650, Tween® 80, Tween® 85, Span® 20, Span® 40, Span® 60, Span® 80, Span® 85; and combinations thereof;
(x) bile acids and their salts, such as sodium cholate, sodium salts of glycolic acids and taurocholic acids, lecithin, deoxycholic acids, and combinations thereof;
(xi) terpenes and terpenoids, including hydrocarbons, such as D-limonene, α-pinene, β-carene; alcohols, such as α-terpineol, terpinen-4-ol and carvol; ketones, such as carvone, pulegone, piperitone, and menthone; oils, such as ylang-ylang, anise, chenopodium, and eucalyptus oils; oxides, such as limonene oxide, α-pinene oxide, cyclohexene oxide, and cyclopentene oxide, and combinations thereof;
(xii) other essential oils (e.g., tea tree oils);
and combinations thereof.

In some embodiments, the penetration enhancer includes at least one of propylene glycol, Tween® 80, Menthol, Coolact® 10, Coolact® P, Coolact® 38, Transcutol® P, and ethanol.

In one embodiment, the active layer is no more than 10 wt. % water, or no more than 3 wt. % water, or no more than 1 wt. % water.

Other excipients which may be present include preservatives, antioxidants, biocides, fungicides.

Numerous additional additives may be present in the described components, which are known in the art. When such additional additives are present the range provided above may be adjusted as needed. In other words, in some embodiments, where additional additives and components are present in the compositions described herein, the percent by weight ranges provided above, may be considered with reference to the main components: TPU, PSA, P(M)AP and active agent.

While not wanting to be bound by any theory it is believed that the polymer blends described herein balance competing goals and properties of drug solubility, drug delivery and physical properties, such as wear properties. For example, while acrylic polymers generally exhibit good solubility for certain drugs, they generally have poor long-term wear properties. Silicone polymers have been used to improve the wear properties of acrylic-containing polymer compositions, silicone may interact with certain drugs, leading to high peel force problems. The present inventors surprisingly found that the use of a thermoplastic polyurethane polymer in combination with a pressure sensitive adhesive has good compatibly with many drugs, achieves desired drug delivery of many drugs, and also provides very good wear properties, without suffering from high peel force problems. Overall, the compositions described herein achieve effective, sustained drug delivery with good physical properties, including wear properties.

### The Backing Layer

The exemplary backing layer 14 is in direct contact with the active layer 12, 12′. In many instances, it is desirable for the backing layer to be impermeable to the active agent (e.g., impermeable to an amphetamine). The backing layer protects the polymer matrix from the environment and prevents loss of the drug and/or release of other components to the environment during use. The backing layer may be any backing layer known in the art for transdermal drug delivery systems.

Example backings include woven and non-woven materials. Backings may be non-occlusive (permeable to air and moisture) or occlusive (air- and water-tight).

Suitable non-woven materials include non-woven polyester felt, polyester-based nonwoven fabrics, such as polyethylene terephthalate spunlaced nonwoven fabrics sold under the tradename Sontara® by Jacob Holm & Sønner Holding A/S (e.g., Sontara® 8001, Sontara® 8004, Sontara® 8007, and Sontara® 8010).

Suitable non-occlusive backings include aromatic polyether-based thermoplastic polyurethanes, available under the tradename Pellethane® from The Lubrizol Corporation, such as Pellethane® 58630-80A (High water swell) and Pellethane® 5863-87A (low/no water swell), which are clear, flexible and breathable backings.

Suitable occlusive backings include polyester film laminates available under the tradename Scotchpak™, from 3M, such as Scotchpak™ 9723, Scotchpak™ 9733, Scotchpak™ 9736, Scotchpak™ 9738, Scotchpak™ 9754, and Scotchpak™ 1012.

### The Release Liner

The release liner 16 is typically located adjacent an opposite face of the active layer 12, IT, as compared to the backing layer. When present, the release liner is removed from the system prior to use to expose the polymer matrix layer 12, 12′ prior to topical application. Materials suitable for use as release liners are well-known known in the art and commercially available, such as polyester release liners, including coated polyester release liners.

### Methods of Manufacture

The compositions described herein can be prepared by methods known in the art. As one step, the active layer compositions described herein can be prepared by methods known in the art, such as blending (mixing) the polymer components in powder or liquid form with an appropriate amount of drug in the presence of an appropriate solvent, such as a volatile organic solvent, optionally with other excipients.

With reference to FIG. 4, a method for forming the device 10 may proceed as follows. The method begins at S100.

At S102, a composition for forming the active layer may be provided. For example, to form the active layer 12, appropriate amounts of one or more polymers, solvent(s) and/or co-solvent(s), and optional excipient(s) are combined and thoroughly mixed together in a vessel. The active agent drug is added to the mixture and agitation is carried out until the drug is uniformly mixed therein.

At S104, the active layer 12 is formed on the release liner 16 (or alternatively, on the backing layer 14). For example, the drug/polymer/solvent mixture may be deposited, e.g., cast, onto a release liner 16 (optionally, at ambient temperature and pressure) followed by evaporation of the volatile solvent(s), for example, at room temperature, slightly elevated temperature, or by a heating/drying step, to form the drug-containing polymer matrix 12 on the release liner.

In one embodiment, the composition is transferred to a coating device where it is coated onto a release liner at a controlled specified thickness. Solvent is removed from the coated composition, such as by passing the composition through an oven in order to drive off all volatile processing solvents.

At S106, the composition is provided with a backing layer 14 (or release liner 16 if the order of assembly is reversed). The backing layer 14 may be applied to the active layer composition prior to complete drying/formation of the active layer.

At S108, the assembly thus formed is optionally post processed, e.g., wound into rolls for storage until final processing and/or cut to an appropriate size and shape for the delivery device, such as by die-cutting from the roll material, and then packaged into pouches.

To form the device 10′, a PSA composition may be deposited, e.g., cast onto a release liner 16 (optionally, at ambient temperature and pressure) to form sub-layer 32 layer followed by deposition of the drug/polymer/solvent mixture, evaporation of the volatile solvent(s), for example, at room temperature, slightly elevated temperature, or by a heating/drying step, to form the drug-containing polymer matrix 30. A backing layer 14 may be applied to the resulting active layer 12′ to form the device.

The order of steps, the amount of the ingredients, and the amount and time of agitation or mixing may be important process variables which will depend on the specific polymers, active agents, solvents and/or cosolvents, and optional excipients used in the composition, but these factors can be adjusted by those skilled in the art. The order in which each method step is performed can be changed if needed without detracting from the invention.

The polymer matrix composition may be applied at a coat weight typical of those used in the art. In some embodiments, the polymer matrix composition is applied at a coat weight of about 10 mg/cm². In some embodiments, the polymer matrix composition is applied at a coat weight of about 12.5 mg/cm². In some embodiments, the polymer matrix composition is applied at a coat weight of about 15 mg/cm².

In accordance with any of the embodiments of compositions described herein, the size of the final product is, in some embodiments, in the range of from 2 cm² to 200 cm², or from 20 cm² to 100 cm² or from 100 cm² to 200 cm2, including 5 cm², 10 cm², 20 cm², 25 cm², 30 cm², 40 cm², 50 cm², 60 cm², 70 cm², 75 cm², 80 cm², 90 cm², 100 cm², 120 cm², 140 cm², 160 cm², 180 cm², 200 cm², and any range made therefrom.

### Methods of Use

The compositions described herein are useful in methods for the topical and/or transdermal delivery of an active agent (cosmetic or pharmaceutical). Thus, the compositions described herein can be used in the treatment of any condition that for which a pharmaceutical is prescribed, available in a pharmacy over the counter or cosmetic.

In some embodiments, the compositions achieve topical and/or transdermal delivery of drug over a period of time of at least 8 hours, including a period of time of at least 8 hours to at least 12 hours, at least 24 hours, or longer. In other embodiments, the compositions achieve topical and/or transdermal delivery of the active agent over a longer period of time, such as over a period of at least 2 days, at least 3 days, at least 4 days, at least 5 days, at least 6 days, at least 7 days, or longer.

The compositions described herein achieve a transdermal flux of active agent (and/or one or more pharmaceutically acceptable salt(s) thereof) that is sufficient to have a therapeutic effect. As used herein, “flux” (also called “permeation rate”) is defined as the absorption of a drug through skin or mucosal tissue, and is described by Fick's first law of diffusion: J=−D(dCm/dx) where J is the flux in g/cm²/sec, D is the diffusion coefficient of the drug through the skin or mucosa in cm²/sec and dCm/dx is the concentration gradient of the drug across the skin or mucosa.

The amount of each chemical component described is presented exclusive of any solvent which may be customarily present in the commercial material, that is, on an active chemical basis, unless otherwise indicated. However, unless otherwise indicated, each chemical or composition referred to herein should be interpreted as being a commercial grade material which may contain the isomers, by-products, derivatives, and other such materials which are normally understood to be present in the commercial grade.

It is known that some of the materials described above may interact in the final formulation, so that the components of the final formulation may be different from those that are initially added. For instance, metal ions can migrate to other acidic or anionic sites of other molecules. The products formed thereby, including the products formed upon employing the composition of the technology described herein in its intended use, may not be susceptible of easy description. Nevertheless, all such modifications and reaction products are included within the scope of the technology described herein; the technology described herein encompasses the composition prepared by admixing the components described above.

### EXAMPLES

The embodiments described herein may be better understood with reference to the following non-limiting examples.

### Abbreviations Used:

CMC.Na=sodium carboxymethylcellulose
CMC=carboxymethylcellulose
BDO=1,4 butanediol (chain extender)
H12MDI=dicyclohexylmethane-4,4′-diisocyanate (polyisocyanate)
PAA=polyacrylic acid
PIB=polyisobutylene
PVA=polyvinyl alcohol
EVA=poly ethylenevinylacetate
EtOH=ethanol
EtOAc=ethyl acetate
PEG-300 is a polyethylene glycol with an average molecular weight of approximately 300 daltons (a polyether C)
PEG-8000 is a polyethylene glycol with an average molecular weight of approximately 8000 daltons (a polyether polyol A)
PEG-1450 is a polyethylene glycol with an average molecular weight of approximately 1450 daltons (a polyether polyol B2)
PTMG 1000=Poly(tetramethylene ether) glycol with an average molecular weight of approximately 1000 daltons
Poly-G=Poly-G®55-56 polyol (Monument Chemical), which is an ethylene oxide-capped diol with a nominal molecular weight of 2,000 (55-56 wt. %) (polyol B1). The ethylene oxide cap yields a high primary hydroxyl content which enhances reactivity.
SD=Standard Deviation

### Components Used

1. Thermoplastic Polyurethanes

The following TPUs are considered (the first number designates the TPU type, as discussed above, such that TPU 1-1 is a TPU of type TPU1):

| | | Polyol | Polyol B2 | | Isocyanate | Chain Extender |
|---|---|---|---|---|---|---|
| | Polyol A | B1 | (PEG-1450 | Polyol C | (H12MDI, except | (BDO except |
| TPU | (PEG-8000) | (Poly-G) | except as noted) | (PEG-300) | as noted) | as noted) |
| TPU 1-1 | 24.73 | 57.82 | | 1.68 | 14.39 | 0.84 |
| TPU 1-2 | 24.7 | | 57.7 | 0.99 | 15 | 0.99 |
| TPU 1-3 | 24.7 | | 57.7 | 0.75 | 15 | 1.12 |
| TPU 1-4 | 24.7 | | 57.7 | 0.6 | 15 | 1.2 |
| TPU 1-5 | 26.29 | | 61.25 | 0.6 | HDI 10.13 | 1.21 |
| TPU 1-6 | 24.49 | | 57.07 | 0.65 | 14.98 | Decanediol 2.27 |
| TPU 2-1 | 90.5 | | | | 7.4 | 1.5 |
| TPU 2-2 | 86.5 | | | | 10.4 | 2.5 |
| TPU 2-3 | 82.5 | | | | 13.3 | 3.6 |
| TPU 2-4 | 82.5 | | | 7.1 | 9.26 | |
| TPU 3-1 | | | 10 (+36 | | 40.5 | 13.5 |
| | | | PTMG 1000) | | | |
| TPU 3-2 | | | 70 | | 22.3 | 7.6 |

TPU 1-1 (Melt Index 2.8) is available from The Lubrizol Corporation (see, for example, Qiong Tang, et al., “Transform™ Polymer Films: Rapid Drug Delivery Hydrogels.”

2. Pressure Sensitive Adhesives (PSA)

Duro-Tak®87-2516: An acrylates copolymer containing vinyl acetate, Polybutyl titanate crosslinker, having —OH functional groups and a viscosity of 4350 mPa·s, solids content 41.5%, dissolved in a blend of ethylacetate and ethanol.

Duro-Tak®387-2051: An acrylates copolymer containing vinyl acetate, no crosslinker, having —COOH functional groups and a viscosity of 4000 mPa·s, solids content 51.5%, dissolved in ethylacetate.

3. Polymethylacrylate Polymer (P(M)AP)

Eudragit®E100, poly(butyl methacrylate-co-(2-dimethylaminoethyl) methacrylate-co-methyl methacrylate) 1:2:1 copolymer (CAS #24938-16-7).

### Test Measurements

Average tackiness is measured according to ASTM D2979-16 “Standard Test Method for Pressure-Sensitive Tack of Adhesives Using an Inverted Probe Machine.”

180° peel is measured according to ASTM D3330M-04 “Standard Test Method for Peel Adhesion of Pressure-Sensitive Tape,” expressed in N/25 mm.

Residual active agent (e.g., Lidocaine) is measured by HPLC.

In vitro permeation test using synthetic skin: conducted using Hanson vertical diffusion cells. A human skin substitute synthetic membrane, Strat-M® membrane (Millipore-Sigma), is used in the transdermal diffusion testing that is predictive of diffusion in human skin. The following experimental conditions are used for the in vitro permeation testing: Diffusion area: 1.77 cm²; Receiver Volume: 7 mL; Receiver Solution: phosphate buffer saline, pH 7.4; Receiver temperature: 32.5° C.; Sample volume: 0.5 ml which is replaced with 0.5 ml fresh acceptor media.

Absolute and relative Permeation (mg) mouse skin is measured to determine whether the application of the transdermal patch will release the active ingredient. Mouse skin membrane is used for Fentanyl permeation tests. Full skin from 8-week NCRNU mouse (Taconic) is prepared to constant skin thinness via laser abrasion and measured for consistent thickness via light transmission. A cubic plastic container with a cylindric volume reservoir core is used. The acceptor reservoir volume is 20 ml, and stirred via magnetic stir bar and covered with a plastic top. HEPES-Buffer pH 7.4 is used as acceptor medium. The diffusion surface is a 1 cm² aperture placed near the bottom on the side of the cubic container. The diffusion surface is from the receptor to outside assembled in the following manner: The epidermis side of the mouse skin is fixed to the adhesive side of the test patch. The backing of the test patch is secured with double sided tape fixed to foam rubber layer which is secured via adhesive to a plastic cover secured to the cubic plastic container. The test temperature is adjusted to 32° C. using a metal heating block thus corresponding to the surface temperature of in vivo human skin. 1 ml samples are taken at the specified time points and replaced with 1 ml of fresh acceptor medium. The content of fentanyl in the acceptor medium is determined by HPLC.

### Example A: Comparison of Average Tackiness and 180° Peel for Commercial Patches and TPU/PSA Compositions

### Example A1

Exemplary lidocaine patches are prepared as follows:

Mixture A: The acrylic adhesive (Duro-take 387-2516 or Duro-take 387-2501).

Mixture B: TPU is dissolved in solvent at 50° C. and then cooled. For TPU1-1, solvent is ethyl acetate, for TPU 2-1, the solvent is a blend of ethanol/ethyl acetate/toluene.

Mixture B is added to Mixture A and stirred thoroughly until a uniform mixture is obtained. Lidocaine and Coolact® 10 are then added to Mixture A+B and stirred until a uniform mixture is obtained to complete the casting solution. The solvent in the final solution is approximately 80% ethyl acetate and 20% ethanol. To adjust total solids solvent may be added.

The casting solution is knife cast onto silicone coated PET 3 mil release liner (Technicote, Inc.) and all solvents are removed by evaporation.

A backing film of Sontara® 8007 is laminated on the drug-containing active layer. The obtained laminated sheet is cut into rectangles of approximately 6.3 cm×7.6 cm. The complete patch is immediately pouched and heat sealed within PET/AL/PAN packaging material.

A set of commercially-available lidocaine patches were obtained for comparison with the exemplary patches. The comparative examples are summarized in TABLE 1 and are referred to as patches L1-L4. Patches L1, L2, and L3 are hydrated. Patches L4 and L5 are solvent cast. Hydrated and solvent-based formulations have very different adhesion and release properties but both types are included here for comparison. Patches L1 and L4 are prescription-only in the US, while L2, L3, and L5 are available over the counter. Examples 6-16 are prepared using different ratios of TPU:PSA. Duro-Tak®87-2516 is used as the PSA and TPU 1-1 as the TPU. TABLE 2 provides details of Examples 6-13 and results obtained for average tackiness and 180° peel. PSA content of the commercial patches (Ex. 1-6) has not been determined.

TABLE 1
Composition of Commercial Lidocaine patches

| | Ex. 1 | Ex. 2 | Ex. 3 | Ex. 4 | Ex. 5 |
|---|---|---|---|---|---|
| Patch | L1 | L2 | L3 | L4 | L5 |
| Lidocaine Conc. | 5% | 4% | 4% | 5% | 4% |
| Lidocaine Load | 700 | 560 | 246 | 140 | 29 |
| (mg) | | | | | |
| Thickness with | 1540 | 1420 | 900 | 270 | 670 |
| liner (μm) | | | | | |
| Form | Thick | Thick | Hydrated | Thin | Thick |
| | hydrated | hydrated | fabric | polymer | fabric |
| | fabric | fabric | | film | |
| Backing | Non-woven | Non-woven | Non-woven | Pigmented PET | Non-woven |
| | polyester felt | polyester felt | polyester felt | | fabric |
| Adhesive | PAA/PVA/ | PAA/PVA/ | Cellulose | PIB | PIB |
| | CMC/Al/H₂O | Al/H₂O | gum/silica/ | | |
| | | | PAA/Al/H₂O | | |
| Liner | PET/silicone | PET/silicone | PET/silicone | PET/silicone | PET/silicone |

Results

| | | | | | |
|---|---|---|---|---|---|
| Residual | 97 ± 2%* | 97 ± 2%** | 97 ± 2%** | 89 ± 4%* | 89 ± 4%** |
| Lidocaine | | | | | |
| Amt Delivered | 18.3* | 16.8** | 74** | 15.4* | 3.2** |
| (mg) | | | | | |

*from prescribing information
**Calculated

TABLE 2
Adhesive properties of Commercial Lidocaine
patches and TPU/PSA Compositions

| | | | Avg | | | |
|---|---|---|---|---|---|---|
| | PSA | TPU | Tackiness | | 180° | |
| Example | (wt. %) | (wt. %) | (N) | SD | Peel | SD |
| 1 (L1) | | | 0.31 | 0.02 | 1.76 | 0.19 |
| 2 (L2) | | | 0.10 | 0.01 | 1.50 | 0.53 |
| 3 (L3) | | | 0.14 | 0.05 | 0.82 | 0.21 |
| 4 (L4) | | | 2.72 | 0.07 | 2.00 | 2.63 |
| 5 (L5) | | | 2.55 | 0.24 | 2.55 | 0.49 |
| 6 | 0 | 100 | 0.26 | 0.03 | 0.00 | 0.00 |
| 7 | 30 | 70 | 0.95 | 0.19 | | |
| 8 | 34.5 | 65.5 | 0.80 | 0.09 | 4.92 | 5.05 |
| 9 | 40 | 60 | 1.88 | 0.19 | 9.38 | 0.81 |
| 10 | 45 | 55 | 2.85 | 0.20 | 5.97/5.80 | 1.37/1.67 |
| 11 | 50 | 50 | 2.94/2.93 | 0.20 | 7.95 | 0.40 |
| 12 | 55 | 45 | 4.11 | 0.28 | 9.93/6.87 | 1.62/0.48 |
| 13 | 60 | 40 | 3.44 | 0.14 | | |
| 14 | 65 | 35 | 5.64 | 0.89 | 13.81 | 3.46 |
| 15 | 76.6 | 23.4 | 6.65 | 0.74 | 12.89 | 0.87 |
| 16 | 100 | 0 | 5.39 | 0.65 | | |

The data in TABLE 2 suggests that the exemplary compositions should provide improved tackiness and/or peel over commercially available compositions, particularly when the PSA is present from 30 or 34 wt. % to 70 or 80 wt. %, i.e., a TPU:PSA ratio of 2:3:1 to 1:4.

### Example A2

A set of commercially-available menthol patches were obtained for comparison with the exemplary patches. The comparative examples are labeled as patches M1-M5 and are all available over-the-counter in the US. TABLE 3 summarizes the details of these patches.

TABLE 3
Commercial menthol patches

| | Ex. 17 | Ex. 18 | Ex. 19 | Ex. 20 | Ex. 21 |
|---|---|---|---|---|---|
| Patch | M1 | M2 | M3 | M4 | M5 |
| Menthol Load | 5 | 5 | 7.5 | 3 | 5 |
| (wt. %) | | | | | |
| Menthol Load | N.A. | 2.107 | 1.02 | 0.451 | N.A. |
| (mg/cm²) | | | | | |
| Other Active | — | — | — | Methyl salicylate | — |
| agents | | | | (10%) | |
| Thickness with | 1320 | 850 | 620 | 660 | 930 |
| liner (μm) | | | | | |
| Form | Hydrated | Hydrated | Non-hydrated | Non-hydrated | hydrated |
| Backing | Nonwoven | Nonwoven | fabric | fabric | Nonwoven |
| | polyester felt | polyester felt | | | polyester fabric |
| Adhesive | Cellulose | Cellulose | PIB | PIB | Polyacrylate/ |
| | gum/acrylate | gum/H₂O | | | CMC•Na/H₂O |
| | copolymer/H₂O | | | | |
| Liner | PET/silicone | PET/silicone | PET/silicone | PET/silicone | PET/silicone |
| Avg Tackiness (N) | 0.11 | 0.15 | 0.37 | 1.07 | 0.22 |
| SD | 0.00 | 0.01 | 0.13 | 0.13 | 0.21 |

As can be seen from TABLE 2 and TABLE 3, the average tackiness of exemplary TPU/PSA compositions compares favorably with the commercial menthol-based samples. The following examples are also prepared to demonstrate the average tackiness, as measured according to ASTM D2979-16. The PSA used in these examples is Duro-Tak®87-2516. The TPU used in these examples is TPU 1-1.

### Example A3

In this Example, skin adhesion, as evaluated according to FDA Draft Guidance for Industry on Assessing Adhesion with Transdermal Delivery Systems and Topical Patches for ANDA's, is evaluated. The adhesion scoring system is listed in TABLE 4 below and the results for the examples are summarized in TABLE 5.

TABLE 4
Scoring system

| Score | Observation |
|---|---|
| 0 | ≥90% adhered (essentially no lift off the skin) |
| 1 | ≥75% to <90% adhered (some edges only lifting off the skin) |
| 2 | ≥50% to <75% adhered (less than half of the patch lifting |
| | off the skin) |
| 3 | >0% <50% adhered but not detached (more than half of the |
| | patch lifting off the skin without falling off) |
| 4 | 0% adhered - patch detached (patch completely off the skin) |

TABLE 5
Observations

| | | |
|---|---|---|
| | | Skin adhesion after |
| PSA | TPU | 12 hrs application (N = 5) |

| Example | (wt. %) | (wt. %) | Score | Observation |
|---|---|---|---|---|
| 22 | — | — | 1 | only some edges |
| | | | | lifting off the skin |
| 13 | 60 | 40 | 0 | essentially no |
| | | | | lift off the skin |
| 11 | 50 | 50 | 0 | essentially no |
| | | | | lift off the skin |

The data suggests that the exemplary compositions should provide improved skin adhesion over commercially available compositions when applied on human skin for 12 hours.

### Example B: Comparison of Release Characteristics for Commercial Lidocaine Patches and Exemplary TPU/PSA Compositions

### Example B1

To investigate the release characteristics of the exemplary compositions, patches with commercially-available formulations according to Examples 1, 3, and 4 from TABLE 1 are compared with a patch according to the exemplary embodiment, Example 23 (having the same adhesive matrix as Example 11 and being loaded with 4% lidocaine, 0.5% polysorbate 80 (polyoxyethylene (20) sorbitan monooleate) and 0.5% propylene glycol in the matrix).

In vitro permeation experiments are conducted with Example 1, 3, 4, and 23 using Hanson vertical diffusion cells and Strat-M® membrane, as described above. Patches are cut into a bigger size than the diffusion area to ensure that the diffusion area through the experiment is covered by the patch. The adhesive side of a patch sample is applied centered to the smooth side of the synthetic membrane, and then the assembly with the rough side of the synthetic membrane facing down is mounted on the top of a vertical cell. During the experiment, the patches with backing facing up are kept non-occlusive to the environment to mimic real applications. At each sampling time point (1, 2, 4 and 6 hour), a volume of 0.5 mL is removed and replaced by 0.5 mL fresh PBS.

6 replicates are tested for all the patch types above. The samples are assayed by high performance liquid chromatography (HPLC). Lidocaine permeation profiles from commercial examples (1, 3, and 4) and patch Example 23 can be differentiated with in vitro permeation test with the synthetic skin membrane. Water based commercial patches with relatively higher lidocaine loading (Examples 1 and 3) show higher lidocaine permeation flux compared to adhesive patches (Example 4 and 23). The Example 23 adhesive matrix, with the exemplary composition, has a higher lidocaine permeation through the membrane over 6 hours than commercial Example 3. The results are summarized in TABLE 6.

TABLE 6
Lidocaine Permeation

| | 1 Hour | | 2 Hours | | 4 Hours | | 6 Hours | |
|---|---|---|---|---|---|---|---|---|
| Ex. | μg/cm² | % | μg/cm² | % | μg/cm² | % | μg/cm² | % |
| 1 | 6.56 (0.74) | 0.13 (0.01) | 19.37 (1.09) | 0.39 (0.02) | 52.00 (5.90) | 1.04 (0.12) | 92.18 (11.70) | 1.84 (0.23) |
| 3 | 3.24 (0.36) | 0.20 (0.02) | 9.18 (2.32) | 0.57 (0.15) | 17.21 (3.06) | 1.06 (0.20) | 28.02 (5.27) | 1.72 (0.33) |
| 4 | 0.39 (0.09) | 0.04 (0.01) | 0.94 (0.17) | 0.09 (0.02) | 3.15 (0.45) | 0.31 (0.04) | 8.08 (0.75) | 0.78 (0.07) |
| 23 | 1.42 (1.17) | 0.17 (0.12) | 3.24 (1.96) | 0.38 (0.20) | 8.21 (4.52) | 0.98 (0.48) | 13.52 (5.90) | 1.60 (0.61) |

(Standard Deviation in parenthesis)

### Example B2

In the following examples aimed at increasing the lidocaine permeation, Examples 24-28 add penetration enhancers to the adhesive matrix of Example 23 at 5 wt. %. (Example 24: polysorbate 80, Example 25: di(ethylene glycol) monoethyl ether (Transcutol®, available from Millipore-Sigma), Example 26: propylene glycol, Example 27: isopulegol (Coolact® P, available from Vantage) and Example 28: methoxypropanediol (Coolact® 10, available from Vantage). Example 29 is a control formulation without penetration enhancers but at the same level of lidocaine loading as the others. The in vitro permeation test is conducted using Hanson vertical diffusion cells, as described above. TABLE 7 shows the lidocaine permeation for these examples.

TABLE 7
Lidocaine Permeation with and without Penetration Enhancers

Lidocaine permeated at 6 hours

| Example | μg/cm² | S.D. | % | S.D. |
|---|---|---|---|---|
| 24 | 17.04 | 2.63 | 2.27% | 0.36% |
| 25 | 20.79 | 1.78 | 2.92% | 0.23% |
| 26 | 33.73 | 3.00 | 4.75% | 0.39% |
| 27 | 33.46 | 6.11 | 4.37% | 0.75% |
| 28 | 94.64 | 11.13 | 10.81% | 1.30% |
| 29 | 13.52 | 3.47 | 1.40% | 0.34% |
| (no enhancer) | | | | |

With the addition of penetration enhancers, the lidocaine permeation is increased by up to 600%. The patch examples that include a penetration enhancer show very good and even an equivalent amount of lidocaine permeation to the best commercially available performance benchmark (Example 1), but the loading of lidocaine in these patches is only 20% of the benchmark patch. These results demonstrate one of the many benefits of the described technology.

### Example C

Additional active agents can be incorporated into patches 10, 10′, as illustrated in TABLE 8. The acrylic adhesive used as the PSA may be Duro-Tak®387-2516.

TABLE 8
Examples of Patches

| | | | PSA | TPU | | Active |
|---|---|---|---|---|---|---|
| Ex. | Patch type | | (wt. %) | (wt. %) | Active agent | (wt. %) |
| 40 | FIG. 1 | Layer 12 | 35 | 35 | (NaproxenNa) | 30 |
| 41 | FIG. 3 | Layer 30 | 35 | 35 | Rivastigmine | 30 |
| | | Layer 32 | 50 | 50 | — | — |
| 42 | FIG. 3 | Layer 30 | 35 | 35 | Rivastigmine or | 30 |
| | | | | | NaproxenNa | |
| | | Layer 32 | 100 | 0 | — | — |

Examples 41-42 are prophetic examples. Replacing the active of Example 42 with a placebo, the adhesion to the skin is very high. Accordingly, adding TPU to the adhesive in layer 32 (as in prophetic Example 41, for example) is expected to provide a more suitable adhesion.

### Example C: Patches Incorporating a Poly(Meth)Acrylate Polymer (P(M)AP) in the Active Layer

Solubility studies are performed in various solvents (ethanol, ethanol/water, isopropanol, ethyl acetate, dioxane, and tetrahydrofuran) to determine suitable solvents for the TPUs. For TPU 2-4, ethanol is selected as the solvent, for TPU 2-1 the selected solvent is a blend of ethanol/ethyl acetate/toluene, and for TPU 1-1, the solvent selected is ethyl acetate, for TPU 3-1 the selected solvent is a blend of ethanol/ethylacetate and THF.

### Method of Making the Transdermal Patch

Mixture A: The acrylic adhesive (Duro-Take 387-2516 or Duro-Take 387-2051 (abbreviated to 2516 and 2051) is blended with a selected TPU and stirred. For example, 13.60 g TPU 2-4 are dissolved in 77.02 g ethanol 96%. The mixture is stirred for 3 hours with a spiral stirrer at 300 rpm to form a solution. 39.84 g (says 3.84 below) of Duro-Take Pressure-sensitive Adhesive (e.g., Duro-Take 387-2051(51.2% in ethyl acetate)) is added and the mixture stirrer with a spiral stirrer at 300 rpm for one hour.

| Mixture A Formulation | Weight [g] |
|---|---|
| Aliphatic polyether thermoplastic | 13.60 |
| polyurethane (TPU 2-4, 2-1, or 1-1) | |
| Pressure-sensitive Adhesive (Duro-Tak ® | 39.84 |
| 387-2051(51.2% in ethyl acetate)) | |
| Ethanol 96% (denatured) | 77.02 |

Mixture B1 (and C1): Fentanyl Solution. 5.5 g Fentanyl are dissolved in 22.0 g Ethanol and are gently stirred at 50 rpm.

| Mixture B1 Formulation | Weight [g] |
|---|---|
| Fentanyl(powder) | 5.50 |
| Ethanol 96%(solvent) | 22.00 |

Mixture B2: TPU1 Stock Solution (15%). 45.0 g TPU1 are dissolved in 255.0 g Ethanol and are stirred at 50-300 rpm overnight.

| Mixture B2 Formulation | Weight [g] |
|---|---|
| TPU1 | 45.00 |
| Ethanol 96% | 255.00 |

Mixture B3: EUDRAGIT® E100 Stock Solution (50%). 40.0 g EUDRAGIT® E 100 are dissolved in 40.0 g ethyl acetate and are stirred at 50-300 rpm overnight.

| Mixture B3 Formulation | Weight [g] |
|---|---|
| Eudragit ® E100 | 40.00 |
| Ethyl acetate | 40.00 |

Mixture B4: Coating solution. 15.2 g EUDRAGIT® E100-Stock Solution 50% are mixed with 23.25 g ethyl acetate and 19.00 g Fentanyl Solution 20%. The mixture is stirred using a spiral stirrer at 200 rpm for 30 min. 18.55 g Duro-Take 387-2051 (51.2% in ethyl acetate) are added. Stirring with a spiral stirrer at 200 rpm is continued for 15 minutes. 114.0 g TPU 2-4 Stock Solution 15% are added. Stirring with a spiral stirrer at 200 rpm is continued for 15 minutes.

| Mixture B3 Formulation | Weight [g] |
|---|---|
| Fentanyl-Solution 20% | 19.00 |
| TPU1 Stock Solution 15% | 114.00 |
| Eudragit ® E100-Stock Solution 50% | 15.20 |
| Duro-Tak ® 387-2051 (51.2% | 18.55 |
| in ethyl acetate) | |
| Ethyl acetate | 23.25 |

Mixture C2: Duro-Take-EUDRAGIT® E100 Stock Solution. 15.57 g EUDRAGIT® E100 are dissolved in 90.95 g Duro-Take 387-2516 (41.9% in ethyl acetate). The solution is stirred overnight at 300 rpm with a T-stirrer.

| Mixture C2 Formulation | Weight [g] |
|---|---|
| Duro-Tak ® 387-2516 (41.9% solids in ethyl acetate) | 90.95 |
| Eudragit ® E100 (solid granules) | 15.57 |

Mixture C3: Coating solution. 16.99 g TPU 2-4 are dissolved in 138.07 g Ethanol 96%. The mixture is stirred for 3 hours with a spiral stirrer at 300 rpm to form a solution. 71.01 g of Duro-Take-EUDRAGIT® E 100 Stock Solution and 26.10 g of Fentanyl solution are added and stirred with a spiral stirrer at 300 rpm for one hour.

| Mixture C3 Formulation | Weight [g] |
|---|---|
| TPU 2-4 | 16.99 |
| Ethanol 96% | 138.07 |
| Duro-Tak ® - EUDRAGIT ® E100 | 71.01 |
| Stock Solution | |
| Fentanyl Solution | 26.10 |

The final solution is approximately 20% ethyl acetate and 80% ethanol.

A transdermal coating machine is used for manufacturing laminates from the solutions. The casting solution is knife cast onto silicone-coated PET release liner (Loparex Primeliner 78 HL) and all solvents are removed by evaporation at 100° C. using a 0.2 m/min coating speed to form the active layer 12. A backing film 14 consisting of a PET film (Scotchpak® 1109) is laminated on the drug in adhesive matrix layer. The obtained laminated sheet is cut into squares of approximately 10 cm². The obtained laminated sheet is cut into squares of approximately 10 cm². The complete patch 20 is immediately pouched and heat sealed within PET/AI/PAN packaging material.

Compositions and results obtained are shown in TABLE 9.

TABLE 9
Composition of dried active layer coating

| | | | | | | | Added Diluent |
|---|---|---|---|---|---|---|---|
| | Fentanyl | E100 | Acrylate | Acrylate | TPU | TPU | Solvent |
| Ex. | (wt. %) | (wt. %) | Type | (wt. %) | Type | (wt. %) | (removed) |
| 51 | 10 | 0 | 2516 | 45 | 2-4 | 45 | EtOH |
| 52 | 10 | 0 | 2051 | 45 | 2-4 | 45 | EtOH |
| 53 | 10 | 15 | 2516 | 45 | 2-4 | 30 | EtOH |
| 54 | 10 | 15 | 2051 | 45 | 2-4 | 30 | EtOH |
| 55 | 9 | 17.9 | 2516 | 43.8 | 2-4 | 29.3 | EtOH |
| 56 | 9 | 17.9 | 2516 | 43.8 | 2-4 | 29.3 | EtOH |
| 57 | 10 | 10 | 2051 | 35 | 2-4 | 45 | EtOH |
| 58 | 10 | 20 | 2051 | 15 | 2-4 | 45 | EtOH/EtOAc |
| 59 | 10 | 45 | — | 0 | 2-4 | 45 | EtOH |
| 60 | 10 | 0 | 2051 | 45 | 1-1 | 45 | EtOH |
| 61 | 10 | 0 | 2051 | 45 | 2-1 | 45 | EtOH |

| | Total | Casting | Active Layer | Adhesive | 72 hr Abs. |
|---|---|---|---|---|---|
| | Solids | Thickness | Dry Thickness | Matrix mg/cm² | Permeation |
| EX. | (wt. %) | (μm) | (μm) | (Target) | (μg) |
| 51 | 22.79 | 250 | 37 | 4.33 | 303.26 |
| 52 | 22.83 | 250 | 33 | 3.88 | 174.47 |
| 53 | 21.5 | 250 | 45 | 5.29 | 452.19 |
| 54 | 22.8 | 250 | 34 | 4.04 | 262.11 |
| 55 | 23 | 200 | 51 | 5.14 | 369.39 |
| 56 | 23 | 155 | 30 | 3.60 | 242.33 |
| 57 | 21.4 | 250 | 36 | 4.02 | 316.75 |
| 58 | 20 | 250 | 36 | 3.96 | 329.23 |
| 59 | | 250 | | | 348.79 |
| 60 | 36.59 | 250 | 91 | 9.79 | 204.34 |
| 61 | 32.96 | 250 | 44 | 5.15 | 214.01 |

The examples illustrate that the 72 hr cumulative release for samples prepared with Duro-Take 387-2516 is higher than for Duro-Take 387-2051. When Eudragit® E100 is present (Examples 53-58), both the Duro-Take 387-2516 and Duro-Take 387-2051 samples show further improvement.

Examples 57-59 also suggest that the amount of Duro-Take 387-2051 has little or no impact on the permeation when Eudragit® E100 is present (although it does affect adhesion). Release is about the same with 0 to 45 wt. % Duro-Take content.

The effect of thickness can be seen in Examples 55 and 56, with the same composition providing higher release at higher thickness.

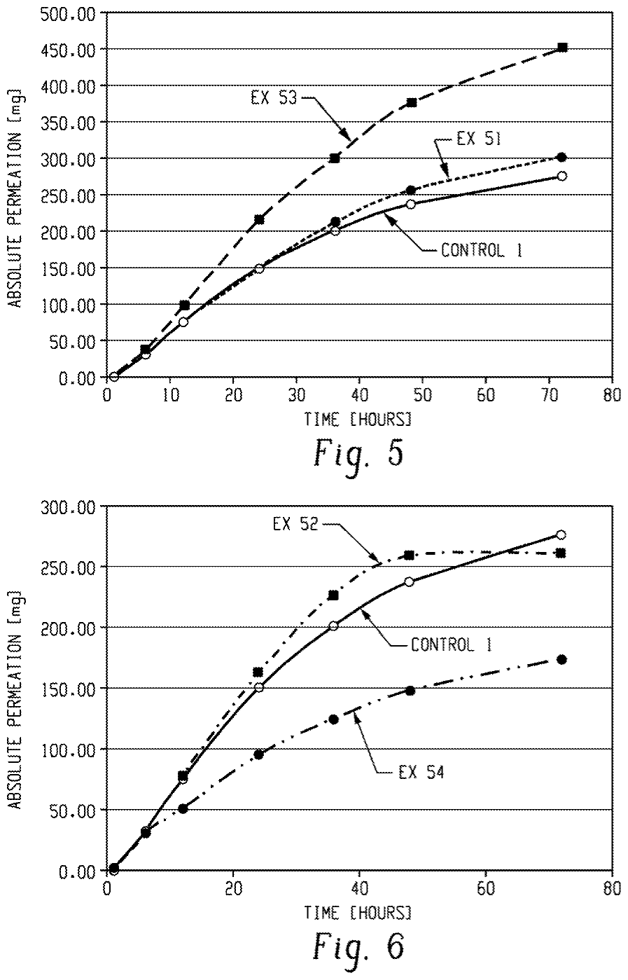

TABLE 10 shows absolute permeation in mg of fentanyl in mouse skin over 72 hours from TPU/Acrylic Adhesives blends 51-56 described above and two reference drug products (Control 1 and Control 2). See also FIGS. 5-8.

TABLE 10
Absolute Permeation of Fentanyl in mg against hour
for mouse skin permeation (average of 3 runs)

| Time, Hr | 1 | 2 | 6 | 12 | 24 | 36 | 48 | 72 |
|---|---|---|---|---|---|---|---|---|
| Ex. 51 | 0.7 | 3.6 | 27.8 | 72.8 | 150.9 | 212.5 | 257.0 | 303.3 |
| SD | 0.61 | 1.98 | 6.83 | 15.61 | 34.82 | 45.32 | 47.01 | 9.64 |
| Ex. 52 | 2.1 | 5.0 | 22.7 | 51.6 | 95.3 | 125.0 | 148.2 | 174.5 |
| SD | 0.70 | 2.08 | 6.95 | 12.22 | 17.47 | 19.27 | 18.41 | 13.99 |
| Ex. 53 | 1.1 | 5.5 | 36.8 | 98.7 | 217.6 | 308.3 | 377.5 | 452.2 |
| SD | 0.54 | 2.07 | 8.15 | 15.64 | 17.47 | 15.03 | 19.88 | 36.96 |
| Ex. 54 | 0.8 | 4.0 | 30.0 | 78.2 | 162.9 | 228.0 | 259.8 | 262.1 |
| SD | 0.72 | 3.03 | 14.13 | 27.04 | 39.75 | 24.24 | 10.30 | 9.56 |
| Ex. 55 | 1.1 | 3.9 | 29.1 | 81.9 | 181.1 | 253.7 | 310.1 | 352.5 |
| SD | 0.59 | 1.54 | 6.12 | 12.30 | 25.60 | 39.72 | 57.87 | 89.29 |
| Ex. 56 | 0.3 | 2.1 | 23.2 | 68.6 | 147.8 | 202.0 | 234.9 | 231.4 |
| SD | 0.27 | 0.46 | 4.12 | 10.80 | 18.88 | 16.88 | 17.40 | 23.30 |
| Control 1 | 0.95 | § | 32.11 | 76.43 | 150.84 | 201.72 | 238.06 | 276.86 |
| Control 2 | 1.24 | 4.44 | 31.20 | 76.48 | 146.73 | 195.12 | 222.69 | 235.42 |

† Data overlaps that of Control F1.
§ No 2-hr data for this example

The differences in composition of the active layer 12 can be seen to have a significant impact on penetration, suggesting that the penetration rate in human skin can be adjusted by tailoring the composition to the desired penetration rate.

In FIG. 5, showing the absolute permeation of Fentanyl from a TPU/Acrylic Adhesives blend against the reference Drug (Control 1). Examples 51 and 53 have the same TPU but different acrylate adhesives. It is considered that the absence of acidic groups in Example 51 may be responsible for improved skin permeation.

FIG. 6 shows absolute permeation of Fentanyl from a TPU/2516 Acrylic Adhesive Blend against reference Drug Product with and without Eudragit® E100. The results suggest that EUDRAGIT® E100 improves skin permeation.

### Example D: Example Fentanyl-Containing Patches Incorporating a Poly(Meth)Acrylate Polymer (P(M)AP) in the Active Layer

A combination of polymers is selected for a fentanyl transdermal patch. The combination enhances the release of fentanyl from the matrix allowing less drug loading. The results suggest that a combination of amine copolymer/TPU/Acrylate with an amine or tertiary amine drug, such as fentanyl, can provide greater release allowing lower loading.

Compositions for a fentanyl transdermal patch are prepared within the ranges as shown in TABLE 11:

TABLE 11
Fentanyl transdermal patch formulations

| Component | Amount (weight %) |
|---|---|
| Fentanyl | 10 |
| Eudragit ® E100 | 10-45 |
| Duro-Tak ® 387-2051 | 0-35 |
| TPU | 29-45 |

TPUs: TPU2-4, TPU2-1, TPU1-1, and TPU3-1 are evaluated.

In the example formulations, the amine drug is paired with an amine copolymer which acts to keep the drug in a non-protonated form as opposed to an acid/base pairing. With Fentanyl it is demonstrated that enhanced release occurs.

The patches are compared with commercial fentanyl patches including an acrylate adhesive patch (denoted F1), a silicone adhesive patch (F2), and a polyisobutylene (PIB) adhesive patch (F3).

TABLE 12 shows characteristic area and loading for these three commercial patches. The expected wear time is 72 hours and the patches should withstand shower and bathing exposure. The silicone and PIB based formulations have adhesion issues and there are multiple reports that they often fall off. The goal is to produce a transdermal patch which matches the fentanyl release profile for the commercial acrylate adhesive patch.

TABLE 12
Example Commercial Fentanyl transdermal patches

| | F1 | | F2 | | F3 | |
|---|---|---|---|---|---|---|
| | Adhesive | | | | | |
| | Acrylate | | Silicone | | Poly(isobutylene) | |
| | Backing | | | | | |
| Dry | Polyester/EVA | | Polyolefin | | | |
| coating | 25 mcg/hr | mg | 25 mcg/h | mg | 25 mcg/hr | |
| (mg/cm²) | 10.5 cm² | 5 | 6.25 cm² | 10 | 10.7 cm² | mg |
| 25 | 10.5 | 4.2 | 6.25 | 2.55 | 10.7 | 2.76 |
| 50 | 21 | 8.4 | 12.5 | 5.10 | 21.4 | 5.52 |
| 75 | 31.5 | 12.6 | 18.75 | 7.65 | 32.1 | 8.27 |
| 100 | 42 | 16.8 | 25 | 10.20 | 42.8 | 11.04 |
| Mg/cm² | | 0.4 | | 0.408 | | 0.257 |
| mg | | 0.08 | | 0.04 | | |
| fentanyl/ | | | | | | |
| mg | | | | | | |
| adhesive | | | | | | |

A Design of Experiments (DOE) of various patch parameters such as thickness, fentanyl content, Eudragit® E100, TPU, and adhesive is conducted to develop a patch with a release profile that matches commercial patch F-1.

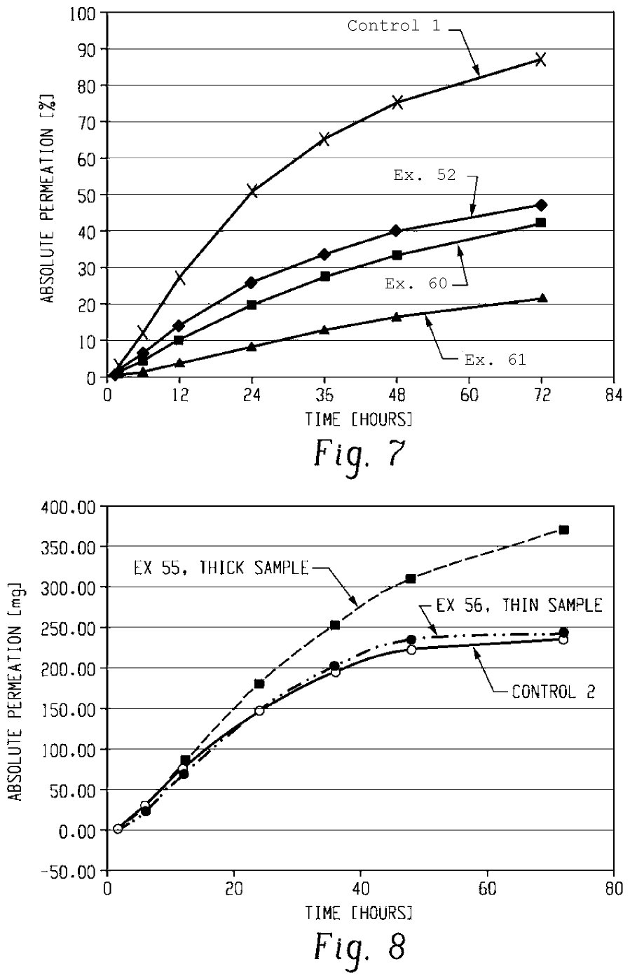

TABLE 13 and FIGS. 7 and 8 show example formulations from the DOE and relative and absolute permeation across mouse skin.

TABLE 13
Exemplary Fentanyl Formulations A and B

| | Formulation A | Formulation B |
|---|---|---|
| Component | Amount (wt. %) | Amount (wt. %) |
| Fentanyl | 9 | 8.6 |
| Eudragit ® E100 | 17.9 | 17.9 |
| Duro-Tak ® 2516 | 43.8 | 43.1 |
| TPU 2-4 | 29.3% | 29.4 |
| Dry thickness (μm) | 51 | 30 |

TABLE 14 shows a comparison of the predicted loading to deliver a similar profile of fentanyl to match a reference drug product. For example, a commercial silicone suspension formulation has a smaller patch with less loading. An acrylate matrix patch requires a higher loading and greater area in order to release the same level of fentanyl. In acrylate matrices, the fentanyl is dissolved in the matrix as opposed to being a suspension or emulsion as is the case in PIB and silicone. In the exemplary formulations, the fentanyl is also dissolved in the matrix.

TABLE 14
Comparison of predicted loading with those of commercial products

| Product | Fentanyl | | Patch Size | | Mg/cm² | Adhesive | P(M)AP |
|---|---|---|---|---|---|---|---|
| Example Formulation A | 3.1 | mg | 10 | cm² | 0.31 | Duro-Tak ® | Eudragit ® |
| | | | | | | 87-2516 | E100 |
| Example Formulation B | 3.5 | mg | 7.5 | cm² | 0.466 | Duro-Tak ® | Eudragit ® |
| | | | | | | 87-2516 | E100 |
| Commercial patches | | | | | | | |
| F-4 Membrane | 2.5 | mg | 10 | cm² | 0.25 | | |
| patch (Reservoir) | | | | | | | |
| F-2 | 2.55 | mg | 6.25 | cm² | 0.408 | Silicone/ | |
| | | | | | | dimethicone | |
| F-5 Emulsion | 2.75 | mg | 8.4 | cm² | 0.327 | | |
| Patch | | | | | | | |
| F-3 Suspension | 2.76 | mg | 10.7 | cm² | 0.25 | PIB | |
| F-6 | 4.13 | mg | 7.5 | cm² | 0.55 | | |
| F-1 | 4.2 | mg | 10.5 | cm² | 0.4 | Acrylates | |
| F-7 | 4.8 | mg | 15 | cm² | 0.32 | | |
| F-8 | 5.78 | mg | 10.5 | cm² | 0.55 | | |
| F-9 | 6.0 | mg | 10 | cm² | 0.6 | | |

API needed: 3 days * 25 μg/h = 1.8 mg

### Example E: Adhesion Testing

High dielectric moisture content skin and oily skin are produced by administration of synthetic sweat and a moisturizing cream, respectively to skin samples. The samples are placed in a humidity- and temperature-controlled room and skin moisture content equilibrated before testing. Skin moisture content is measured by the change in the dielectric constant via capacitance measurement. Patches under study are applied to the treated area, and a panelist completed controlled repeated flexural movement of the treated area over a set time. Skin capacitance comparisons before and after each test, and patch adherence using FDA guidelines for rating adhesion, are correlated. Commercial patches (Ex. 4, Ex. 5, described above), and experimental samples (Ex. 11, described above) are tested. Results are shown in TABLE 15.

For humidity equilibrated skin after the flexural movement test, EX-11 has 100% adherence to skin. Comparative patches showed quite different skin adherence, 90% for Ex. 5 and 44% for Ex. 4. For skin at high dielectric moisture level (i.e., sweaty skin), after flexural movement, EX-11 still shows excellent adhesion to skin (92%), while adhesion for the comparative samples dropped dramatically (Ex. 5, 68%, and Ex. 4, 15%).

Patch adhesion is known to be poor on skin treated with body lotion. Ex. 11 demonstrates better adhesion than the commercial patches using the same high stress flexural model. Patch tackiness to stainless steel results for Ex. 11 and Ex. 4 and Ex. 5 show similar results and in vivo skin adhesion results demonstrate improved adhesion under adverse condition for the Ex. 11 patches. The results from this in vivo method indicate that the example patches provide improved adhesion over current patches, even on sweaty skin and oily skin. Purposeful removal of the experimental transdermal patches is achieved with no adhesive failure or painful peel from the skin.

TABLE 15
Comparison of inventive patch adhesion under
adverse conditions showing improved adhesion.

| | | | | Tack Steel |
|---|---|---|---|---|
| | Baseline skin | Sweaty skin | Oily skin | (N) (ASTM |
| Example | ML 45% | ML 120% | ML 72% | D2979-16) |
| Comparative | 45% | 15 | 23% | 2.72 |
| Ex. 4 | | | | |
| Comparative | 90% | 68 | 20% | 2.55 |
| Ex. 5 | | | | |
| Ex. 11 | 100% | 92% | 67% | 2.94 |

ML = measured skin Moisture Level

### Example F: Tack Properties of TPU/PSA Blends

A series of blends of 50 wt % TPU and 50 wt. % acrylic pressure-sensitive adhesive (Duro-take 387-2516) are formed and tested for tack. The TPU polymer is dissolved in THF and blended with the Duro-take 387-2516, wet coated at 20 mil (approx. 0.5 mm) wet thickness, then dried at 70° C. for 10 minutes. Results are for average tackiness (ASTM D2979-16) are shown in TABLE 16.

TABLE 16
Tack properties fur TPU/Acrylic PSA Blends

| Ex | PSA (wt. %) | TPU (wt. %) | Avg Tackiness (N) | SD |
|---|---|---|---|---|
| 3-1 | 50 | 50 | 1.5 | 0.5 |
| 3-2 | 50 | 50 | 1.9 | 0.3 |
| 2-2 | 50 | 50 | 2.0 | 0.5 |

A series of blends of TPU with silicone-based adhesive (Dow Corning Bio-PSA 7-4502) are prepared. The TPU polymer is dissolved in THF and blended with the silicone adhesive. The mixture is wet coated at 20 mil (approx. 0.5 mm) wet thickness and dried at 70° C. for 10 minutes. Results are for average tackiness (ASTM D2979-16) are shown in TABLE 17.

TABLE 17
Tack properties fur TPU/Silicone PSA Blends

| Ex | PSA (wt. %) | TPU (wt. %) | Avg Tackiness (N) | SD |
|---|---|---|---|---|
| 3-1 | 70 | 30 | 0.9 | 0.3 |
| 3-2 | 70 | 70 | 0.9 | 0.5 |
| 2-2 | 70 | 70 | 1.9 | 0.8 |

Each of the documents referred to above is incorporated herein by reference, including any prior applications, whether or not specifically listed above, from which priority is claimed. The mention of any document is not an admission that such document qualifies as prior art or constitutes the general knowledge of the skilled person in any jurisdiction. Except in the Examples, or where otherwise explicitly indicated, all numerical quantities in this description specifying amounts of materials, reaction conditions, molecular weights, number of carbon atoms, and the like, are to be understood as modified by the word “about.” It is to be understood that the upper and lower amount, range, and ratio limits set forth herein may be independently combined. Similarly, the ranges and amounts for each element of the technology described herein can be used together with ranges or amounts for any of the other elements.

As used herein, the transitional term “comprising,” which is synonymous with “including,” “containing,” or “characterized by,” is inclusive or open-ended and does not exclude additional, un-recited elements or method steps. However, in each recitation of “comprising” herein, it is intended that the term also encompass, as alternative embodiments, the phrases “consisting essentially of” and “consisting of,” where “consisting of” excludes any element or step not specified and “consisting essentially of” permits the inclusion of additional un-recited elements or steps that do not materially affect the basic and novel characteristics of the composition or method under consideration. That is “consisting essentially of” permits the inclusion of substances that do not materially affect the basic and novel characteristics of the composition under consideration.

While certain representative embodiments and details have been shown for the purpose of illustrating the subject technology described herein, it will be apparent to those skilled in this art that various changes and modifications can be made therein without departing from the scope of the subject invention. In this regard, the scope of the technology described herein is to be limited only by the following claims.

1. A transdermal delivery device comprising:
at least one of a backing layer and a removable release liner;
an active layer supported by the at least one of the backing layer and the removable release liner, the active layer comprising:
a polymer matrix,
a therapeutically or cosmetically effective amount of an active agent dispersed in the polymer matrix, and
a pressure sensitive adhesive, the pressure sensitive adhesive being least one of:
incorporated in the polymer matrix, and
adhered to the polymer matrix;
the polymer matrix comprising:
(a) a thermoplastic polyurethane polymer, the thermoplastic polyurethane polymer comprising the reaction product of:
(i) at least one of:
a) a first polyether polyol A having a molecular weight of at least 3000 daltons, and
b) a second polyether polyol B having a molecular weight of no more than 2500 daltons;
(ii) at least one of:
c) a third polyol C having a molecular weight of up to 800 daltons, and
d) a chain extender;
(iii) a polyisocyanate; and
(iv) optionally, a catalyst, and
(b) optionally, a poly(meth)acrylate polymer.

2. The device of claim 1, wherein the pressure sensitive adhesive is at least 20 wt. % of the polymer matrix.

3. The device of claim 1, wherein the thermoplastic polyurethane polymer is at least 20 wt. % of the polymer matrix.

4. The device of claim 1, wherein the pressure sensitive adhesive is incorporated in the polymer matrix.

5. The device of claim 1, wherein the pressure sensitive adhesive is in a layer which is in contact with a layer containing the polymer matrix.

6. The device of claim 1, wherein the pressure sensitive adhesive is selected from the group consisting of acrylic-based pressure sensitive adhesives and silicone-based pressure sensitive adhesives.

7. The device of claim 1, wherein the pressure sensitive adhesive comprises at least one vinyl acetate acrylic pressure sensitive adhesive.

8. The device of claim 1, wherein the poly(meth)acrylate polymer is incorporated in the polymer matrix.

9. The device of claim 8, wherein the poly(meth)acrylate polymer is selected from the group consisting of:
poly(butyl methacrylate-co-(2-dimethylaminoethyl) methacrylate-co-methyl methacrylate);
poly(methacrylic acid-co-methyl methacrylate);
poly(ethyl acrylate-co-methyl methacrylate-co-trimethylaminoethyl methacrylate chloride);
poly(methacrylic acid-co-methyl methacrylate);
poly(methacrylic acid-co-ethyl acrylate); and
mixtures thereof.

10. The device of claim 8, wherein a ratio by weight of poly(meth)acrylate polymer:thermoplastic polyurethane polymer in the polymer matrix is at least 1:100.

11. The device of claim 8, wherein the poly(meth)acrylate is a cationic copolymer derived from dimethylaminoethyl methacrylate, butyl methacrylate, and methyl methacrylate.

12. The device of claim 8, wherein the poly(meth)acrylate is at least 2 wt. % of the polymer matrix.

13. The device of claim 1, wherein the active layer is no more than 10 wt. % water.

14. The device of claim 1, wherein the thermoplastic polyurethane polymer has a hard segment content of at least 4 wt. % and a soft segment content of at least 90 wt. %.

15. The device of claim 1, wherein the chain extender is selected from 1,4-butanediol, 1,10-decanediol, and combinations thereof.

16. The device of claim 1, wherein at least one of:
the first polyether polyol A has a molecular weight of at least 4000 daltons;
the second polyether polyol B, when present, has a molecular weight of no more than 2000 daltons;
a ratio of the molecular weight of the first polyether polyol to the second polyether polyol, when the second polyether polyol is present, is at least 1.4:1;
a ratio by weight of the second polyether polyol to the first polyether polyol, when the second polyether is present, is at least 1:1; and
the chain extender has a molecular weight of up to 300 daltons.

17. (canceled)

18. The device of claim 1, wherein the active layer has a thickness of up to 1 mm.

19. (canceled)

20. The device of claim 1, wherein the active agent is selected from the group consisting of agomelatine, amphetamine, scopolamine, levonorgestrel, ethinyl estradiol, levonorgestrel acetate, testosterone, methylphenidate, enalaprilat ethyl ester, capsaicin, clonidine, ketoprofen, nicotine, lidocaine, lidocaine HCL, fentanyl, rivastigmine, pharmaceutically acceptable salts, prodrugs, and/or derivatives thereof, and combinations thereof.

21. The device of claim 1, wherein the thermoplastic polyurethane polymer comprises the reaction product of:
(i) the first polyether polyol A, which comprises a poly(ethylene glycol) with an average molecular weight of 7000-9000 daltons;
(ii) the second polyether polyol B, which comprises a polyether copolymer diol with polypropylene glycol and polyethylene oxide segments and an average molecular weight of about 2000 daltons;
(iii) the at least one of the third polyol and the chain extender; and
(iv) the polyisocyanate.

22. (canceled)

23. (canceled)

24. (canceled)

25. (canceled)

26. (canceled)

27. (canceled)

28. The device of claim 1, wherein the active layer is adhered to the backing layer and/or wherein the active layer is adhered to one of the adhesive layer and the release liner.

29. (canceled)

30. A method for the transdermal delivery of an active pharmaceutical ingredient, comprising topically applying the device of claim 1, after removal of the release liner, where present, to the skin of a subject in need thereof.

31. A method for the manufacture of a transdermal delivery device, comprising:
(I) forming a composition comprising:
(a) a therapeutically or cosmetically effective amount of an active pharmaceutical and/or cosmetic ingredient;
(b) optionally, a poly(meth)acrylate polymer;
(c) a pressure sensitive adhesive; and
(d) at least 20 wt. % of a thermoplastic polyurethane polymer, the thermoplastic polyurethane polymer comprising the reaction product of:
(i) at least one of:
a) a first polyether polyol A having a molecular weight of at least 3000 daltons, and
b) a second polyether polyol B having a molecular weight of no more than 2500 daltons;
(ii) at least one of:
c) a third polyol C having a molecular weight of up to 800 daltons, and
d) a chain extender;
(iii) a polyisocyanate; and
(iv) optionally, a catalyst;
(II) forming a multilayer structure in which the composition forms a layer intermediate a backing layer and a release liner, whereby the pressure sensitive adhesive is present in at least one of the active layer and a separate layer which spaces the active layer from the release liner.

32. A composition for the delivery of an active pharmaceutical ingredient in the form of a flexible finite device for topical application, the composition comprising:
a polymer matrix comprising:
at least 20 wt. % of a thermoplastic polyurethane polymer, and
at least 30 wt. % of a pressure sensitive adhesive;
at least 2 wt. % of a poly(methacrylate); and
a therapeutically or cosmetically effective amount of an active pharmaceutical or cosmetic ingredient dispersed in the polymer matrix,
wherein said thermoplastic polyurethane polymer comprises the reaction product of:
(i) a first polyether polyol having a molecular weight of at least 3000 daltons;
(ii) a second polyether polyol having a molecular weight of no more than 2500 daltons;
(iii) at least one of a third polyol having a molecular weight of up to 800 daltons and a chain extender;
(iv) a polyisocyanate; and
(v) optionally, a catalyst.

33. A transdermal delivery device, comprising:
(i) a backing layer;
(ii) an integrated matrix comprising:
(a) a therapeutically or cosmetically effective amount of an active pharmaceutical or cosmetic ingredient;
(b) 20 to 70 wt. % of a thermoplastic polyurethane polymer, wherein said thermoplastic polyurethane polymer comprises the reaction product of: (i) a first polyether polyol having a molecular weight of at least 3000 daltons; (ii) optionally, a second polyether polyol having a molecular weight of no more than 2500 daltons; (iii) at least one of: a third polyol having a molecular weight of up to 800 daltons, and a chain extender; (iv) a polyisocyanate; and (v) optionally, a catalyst.
(c) 30 to 80 wt. % of a pressure sensitive adhesive;
wherein said combined adhesive and drug-containing carrier layer, has a first surface and a second surface, and said first surface of said integrated matrix is directly affixed to a surface of said backing layer; and
(iii) a removable release liner;
wherein said removable release liner is directly affixed to said second surface of said integrated matrix.
